(19) United States
(12) Patent Application Publication
(10) Pub. No.: US 2024/0423724 A1
(43) Pub. Date: Dec. 26, 2024

(54) REGISTRATION AND REGISTRATION VALIDATION IN IMAGE-GUIDED SURGERY

(71) Applicant: AUGMEDICS LTD., Yokneam Illit (IL)

(72) Inventors: Stuart WOLF, Yokneam (IL); Nissan ELIMELECH, Beerotaim (IL)

(21) Appl. No.: 18/684,756

(22) PCT Filed: Aug. 25, 2022
(86) PCT No.: PCT/IB2022/057965

§ 371 Date: Feb. 19, 2024

Related U.S. Application Data
(60) Provisional application No. 63/237,205, filed on Aug. 26, 2021.

(51) Int. Cl. A61B 34/20; A61B 34/10; A61B 90/00

(52) U.S. Cl. CPC A61B 34/20; A61B 34/10; A61B 90/361; A61B 2034/105; A61B 2034/107; A61B 2090/363; A61B 2090/376

(57) ABSTRACT

Systems and methods for facilitating registration and validation of registration and/or transformation in connection with image-guided surgery or medical procedures are disclosed. The systems and methods may be used in connection with devices or systems including augmented reality near-eye displays.

### CROSS-REFERENCE TO RELATED APPLICATION

The present application claims the benefit of U.S. Provisional Application 63/237,205, filed Aug. 26, 2021, the entire content of which is incorporated herein by reference.

### FIELD

The present disclosure relates generally to image-guided surgery or intervention, and specifically to systems and methods for use of augmented reality in image-guided surgery or intervention and/or to systems and methods for use in computer assisted navigation during surgery or other medical intervention or diagnostic procedures.

### BACKGROUND

In an image-guided surgical procedure, images of the patient's anatomy, and of tools used in the procedure, may be acquired. Images may be then presented on one or more computer monitors and/or on a screen of a headset of an augmented reality system used by a surgeon performing the procedure. Some procedures involve operations on an entity of the patient that is initially not visible, such as a bone of the patient, and images of these entities may be presented, inter alia, during the procedure.

Applicant's own work has demonstrated that in order for the presented images of the anatomy and the tools to align one with the other and for the presented images of the anatomy and/or the tools to align with the actual scene including the patient actual anatomy. the images may be referenced, directly or indirectly, to a common fiducial marker. In an augmented reality system, this enables the images to be correctly presented on a screen of the system, independently of how the objects themselves may be viewed. The referencing is a two-stage process: initially, the fiducial marker is registered with an element, such as a bone, of the patient. Subsequently, the marker is tracked, so that relative motion between the surgeon and the patient can be adjusted for in the image presentation.

For example, Applicant's prior systems for image-guided surgery have been effective in registering a fiducial marker, and then tracking the marker. For example, U.S. Pat. No. 9,928,629, U.S. Patent Application 2021/0030511, U.S. Patent Application 2021/0161614, and PCT Patent Application PCT/IB2022/056212, each of which are incorporated herein by reference, describe a system having a fiducial marker (e.g., a patient marker) which is fixedly attached (e.g., via an anchoring device such as a clamp or a pin) to the spine of a patient. The marker has radiopaque elements, or is registered to a registration marker having such elements, which, by being imaged when attached to the patient in a computerized tomography fluoroscope, enable the marker to be referenced to the patient. The marker may also have optical elements, enabling the marker to be tracked using optical radiation reflected from the marker, during a procedure on the patient.

U.S. Pat. No. 10,463,434, which is incorporated herein by reference, describes devices and methods for facilitating registration and calibration of surface imaging systems. Tracking marker support structures are described that include one or more fiducial reference markers, where the tracking marker support structures are configured to be removably and securely attached to a skeletal region of a patient. Methods are provided in which a tracking marker support structure is attached to a skeletal region in a pre-selected orientation, thereby establishing an intraoperative reference direction associated with the intraoperative position of the patient, which is employed for guiding the initial registration between intraoperatively acquired surface data and volumetric image data. In other example embodiments, the tracking marker support structure may be employed for assessing the validity of a calibration transformation between a tracking system and a surface imaging system. Example methods are also provided to detect whether or not a tracking marker support structure has moved from its initial position during a procedure.

US Patent Application Publication 2019/0046272, which is incorporated herein by reference, describes a method including receiving a computerized tomography (CT) image of voxels of a subject's head, and analyzing the image to identify respective locations of the subject's eyes in the image, so defining a first line segment joining the respective locations. The method includes identifying a voxel subset overlaying bony sections of the head, lying on a second line segment parallel to the first line segment and on a third line segment orthogonal to the first line segment. A magnetic tracking system configured to measure positions on the subject's head is activated, and a probe, operative in the system, is positioned in proximity to the bony sections to measure positions of a surface of the head overlaying the bony sections. A correspondence between the positions and the voxel subset is formed, and a registration between the CT image and the magnetic tracking system is generated in response to the correspondence.

U.S. Pat. No. 10,499,997, which is incorporated herein by reference, describes system and methods for surgical navigation providing mixed reality visualization. The mixed reality visualization depicts virtual representations in conjunction with real objects to provide improved visualization to users.

U.S. Pat. No. 10,842,461, which is incorporated herein by reference, describes a system and method of checking registration for a surgical system, the surgical system including fiducials and tracking markers. The method may include: using the fiducials and the tracking markers to register a three-dimensional (3D) imaging space of the surgical system with a 3D tracking space of the surgical system; using a tracking fixture of an X-ray imaging system to register an X-ray imaging space of the X-ray imaging system to the 3D tracking space; obtaining a two-dimensional (2D) X-ray image corresponding to the 3D tracking space; identifying a point of interest in the 2D X-ray image; determining a vector in the 3D tracking space that passes through the point of interest; and/or evaluating the registration of the 3D imaging space with the 3D tracking space based on a location, an orientation, or the location and the orientation of the vector in the 3D tracking space.

### SUMMARY

Embodiments of the present disclosure provide, for example, systems and methods for registration and validation of registration in connection with image-guided surgery or medical procedures. The systems and methods of the disclosure each have several innovative aspects, no single one of which is solely responsible for the desirable attributes disclosed herein.

In some embodiments, a method comprises obtaining a transformation for registering a representation of internal anatomy of a subject with a coordinate system with respect to which a fiducial, which is coupled to the subject, is tracked, by applying the transformation: computing initial coordinates, relative to the representation, of one or more landmarks on skin of the subject, facilitating a procedure on the subject by displaying the representation in registration with the coordinate system, and computing subsequent coordinates of the landmarks relative to the representation; computing one or more distances between the subsequent coordinates and the initial coordinates; and in response to the distances, generating an output.

In some embodiments, the fiducial is tracked by identifying the fiducial in tracking images acquired by a camera.

In some embodiments, the procedure is performed by a physician viewing the subject through a near-eye display of a head-mounted display device, and wherein displaying the representation comprises displaying the representation on the near-eye display so as to augment the view of the physician.

In some embodiments, a distance from the fiducial to each of the landmarks is at least ten cm.

In some embodiments, the fiducial is a first fiducial, wherein the procedure is performed using a tool, wherein a second fiducial, which is tracked with respect to the coordinate system, is coupled to the tool, and wherein displaying the representation comprises displaying the representation with an overlaid virtual representation of the tool at a portion of the representation corresponding to a location of the tool.

In some embodiments, displaying the representation comprises displaying the representation with respective virtual representations of the landmarks at the initial coordinates, respectively, and wherein generating the output comprises modifying at least some of the virtual representations of the landmarks.

In some embodiments, generating the output comprises generating the output in response to one or more of the distances exceeding a predefined threshold.

In some embodiments, the output indicates that those of the landmarks corresponding to those of the distances that exceed the threshold should not be used for subsequent validation of the transformation.

In some embodiments, generating the output comprises selecting one or more of the landmarks corresponding to those of the distances that do not exceed the threshold, and generating the output so as to indicate that the selected ones of the landmarks should be used for subsequent validation of the transformation.

In some embodiments, each of the selected ones of the landmarks is farther from a closest one of unselected ones of the landmarks than is any other one of the unselected ones of the landmarks.

In some embodiments, generating the output comprises generating the output so as to indicate that one or more of the landmarks corresponding to those of the distances that exceed the threshold may have locally shifted.

In some embodiments, the threshold is a first threshold, wherein the method further comprises computing respective initial pairwise distances between pairs of the landmarks, and wherein generating the output so as to indicate that one or more landmarks may have locally shifted comprises computing respective subsequent pairwise distances between the pairs, and generating the output so as to indicate that the local shift may have occurred in response to a magnitude of a difference between one of the subsequent pairwise distances and a corresponding one of the initial pairwise distances exceeding a second predefined threshold.

In some embodiments, the output indicates that the transformation may have become invalid.

In some embodiments, the fiducial is a first fiducial, wherein a second fiducial, which is coupled to a tool, is tracked with respect to the coordinate system while the tool contacts different respective ones of the landmarks, and wherein computing the subsequent coordinates comprises in response to the tracking of the second fiducial, computing respective base coordinates of the landmarks, and computing the subsequent coordinates by transforming the base coordinates per the transformation.

In some embodiments, the computing of the one or more distances between the subsequent coordinates and the initial coordinates comprises for each subsequent coordinate, computing a distance between the subsequent coordinate and each of the initial coordinates.

In some embodiments, a landmark is determined valid if a distance between its subsequent coordinates and initial coordinates does not exceed a predefined threshold.

In some embodiments, the computing of the initial exceeding a predefined threshold coordinates of the landmarks and the computing of the subsequent coordinates of the landmarks are performed are performed according to instructions provided by a user.

In some embodiments, the landmarks are uniquely identified by a characteristic and wherein the method further comprises storing the initial coordinates of each landmark in association with its uniquely identifying characteristic.

In some embodiments, for each landmark subsequent coordinates, a user input indicating the corresponding landmark initial coordinates.

In some embodiments, each landmark comprises one or more uniquely identifying retroreflective elements.

In some embodiments, a method for tracking a transformation during a medical procedure, the transformation registering a representation of internal anatomy of a subject with a coordinate system with respect to a fiducial, the fiducial being coupled to the subject, the method comprising determining initial coordinates of one or more landmarks relative to the representation, the one or more landmarks being disposed on skin of the subject, displaying the representation in registration with the coordinate system, determining subsequent coordinates of the one or more landmarks relative to the representation, determining one or more distances between the subsequent coordinates and the initial coordinates, and determining whether the registration is valid based at least in part on the one or more distances.

In some embodiments, the fiducial is tracked by identifying the fiducial in tracking images acquired by a camera.

In some embodiments, the medical procedure is performed by a physician viewing the subject through a near-eye display, and wherein displaying the representation comprises displaying the representation on the near-eye display so as to augment the view of the physician.

In some embodiments, a distance from the fiducial to each of the one or more landmarks is at least ten cm.

In some embodiments, the fiducial is a first fiducial, wherein the procedure is performed using a tool, wherein a second fiducial, which is tracked with respect to the coordinate system, is coupled to the tool, and wherein displaying the representation comprises displaying the representation with an overlaid virtual representation of the tool at a portion of the representation corresponding to a location of the tool.

In some embodiments, displaying the representation comprises displaying the representation with respective virtual representations of the one or more landmarks at the initial coordinates, respectively, and wherein determining whether the registration is valid comprises modifying at least some of the virtual representations of the landmarks.

In some embodiments, determining whether the registration is valid comprises determining whether the one or more distances exceed a predefined threshold.

In some embodiments, the one or more landmarks corresponding to the one or more distances that exceed the predefined threshold are not used for subsequent validation of the transformation.

In some embodiments, the one or more landmarks corresponding to the one or more distances that do not exceed the predefined threshold are used for subsequent validation of the transformation.

In some embodiments, identifying the one or more landmarks corresponding to the one or more distances that exceed the predefined threshold may have locally shifted.

In some embodiments, the predetermined threshold is a first threshold, and wherein identifying the one or more landmarks that may have shifted comprises determining respective initial pairwise distances between pairs of the one or more landmarks, determining respective subsequent pairwise distances between the pairs of the one or more landmarks, determining a magnitude of a difference between one of the subsequent pairwise distances and a corresponding one of the initial pairwise distances, and comparing the magnitude to a second predefined threshold.

In some embodiments, the fiducial is a first fiducial, wherein a second fiducial, which is coupled to a tool, is tracked with respect to the coordinate system while the tool contacts different respective ones of the one or more landmarks, and wherein determining the subsequent coordinates comprises in response to the tracking of the second fiducial, determining respective base coordinates of the one or more landmarks and determining the subsequent coordinates based at least in part on the one or more distances.

In some embodiments, determining the one or more distances between the subsequent coordinates and the initial coordinates comprises for each subsequent coordinate, determining a distance between the subsequent coordinate and each of the initial coordinates.

In some embodiments, the one or more landmarks are determined valid if a distance between its subsequent coordinates and initial coordinates does not exceed a predefined threshold. In some embodiments, the one or more landmarks are uniquely identified by a characteristic, and wherein the method further comprises storing the initial coordinates of the one or more landmarks in association with its uniquely identifying characteristic.

In some embodiments, the one or more landmarks comprise one or more uniquely identifying retroreflective elements.

In some embodiments, a system for tracking a transformation during a medical procedure, the transformation registering a representation of internal anatomy of a subject with a coordinate system with respect to one or more fiducial objects coupled to the subject, the system comprising a head-mounted display device comprising a near-eye display and a tracking system, a plurality of landmarks forming the one or more fiducial objects, the plurality of landmarks configured to be disposed on skin of the subject in proximity to a site of the medical procedure, and one or more processors, that upon execution of program instructions stored on a non-transitory computer-readable medium determine initial coordinates of the plurality of landmarks relative to the representation based on one or more images received by the tracking system of the plurality of landmarks and the site of the medical procedure, display the representation in registration with the coordinate system on the near-dye display of the head-mounted device, determine subsequent coordinates of the plurality of landmarks relative to the representation, determine one or more distances between the subsequent coordinates and the initial coordinates, and determine whether the registration is valid based at least in part on the one or more distances.

In some embodiments, the head-mounted display device comprises a pair of glasses.

In some embodiments, the tracking system comprises an infrared camera.

In some embodiments, the tracking system further comprises a projector configured to project infrared light toward the site of the medical procedure.

In some embodiments, the plurality of landmarks comprise registration markers.

In some embodiments, the plurality of landmarks comprise adhesive stickers.

In some embodiments, the plurality of landmarks comprise one or more uniquely identifying retroreflective elements.

In some embodiments, the plurality of landmarks are disposed in a random pattern on the skin of the subject.

In some embodiments, the plurality of landmarks comprise one or more radiopaque elements.

In some embodiments, a fiducial object comprises a radiotransparent plate having a first surface coated with an optical retroreflector, and a second surface opposite the first surface, a radiopaque element incorporated within the radiotransparent plate, and an adhesive layer, formed on the second surface, configured to removably adhere to skin of a human subject.

In some embodiments, the radiopaque element is a preset distance from the first surface.

In some embodiments, the optical retroreflector is radiotransparent.

In some embodiments, the radiopaque element comprises a radiopaque bead having a symmetrical shape.

In some embodiments, a fiducial marker comprises a flexible sheet, having a first sheet surface and a second sheet surface opposite the first sheet surface, a plurality of fiducial objects, each fiducial object comprising a radiotransparent plate having a first plate surface coated with an optical retroreflector, and a second plate surface, opposite the first plate surface, affixed to the first sheet surface, a radiopaque element incorporated within the radiotransparent plate, and an adhesive layer, formed on the second sheet surface, configured to removably adhere to skin of a human subject.

In some embodiments, the plurality of fiducial objects are affixed to the first sheet surface in a preset pattern.

In some embodiments, a fiducial marker comprises a plurality of fiducial objects, each fiducial object comprising a radiotransparent plate having a first surface coated with an optical retroreflector, and a second surface opposite the first surface, a radiopaque element incorporated within the radiotransparent plate, and an adhesive layer, formed on the second surface, configured to removably adhere to skin of a human subject.

In some embodiments, a method for registering a plurality of fiducial objects, individually attached to the skin of a patient, with the patient, each of the fiducial objects comprising a radiopaque element, the method comprising accessing a fluoroscopic image of the fiducial objects, identifying, in each of the fiducial objects, respective locations of the radiopaque element therein, and in response to the identified respective locations, formulating a vector between a selected point of the fiducial objects and the patient, so as to register the fiducial objects with the patient.

In some embodiments, the vector is between a centroid of the fiducial objects and a vertebra of the patient.

In some embodiments, the vector is between a selected one of the fiducial objects and a point in a fluoroscopic scan providing the fluoroscopic image.

In some embodiments, a method for tracking a plurality of fiducial objects individually attached to the skin of a patient, each of the fiducial objects comprising a radiopaque element therein and a retroreflector thereon, the method comprising accessing a fluoroscopic image of the fiducial objects, identifying, in each of the fiducial objects, respective locations of the radiopaque elements therein, in response to the identified respective locations, defining a first shape of the attached fiducial objects, acquiring an optical image of the fiducial objects in response to optical radiation transmitted from a head mounted display and identifying the retroreflectors in the image, formulating a second shape of the attached fiducial objects in response to the identified retroreflectors, and when the second shape corresponds to the first shape, using the identified retroreflectors to track the plurality of fiducial objects in a frame of reference of the head mounted display.

In some embodiments, defining the first shape comprises generating a set of local vectors between the identified locations of the radiopaque elements.

In some embodiments, formulating the second shape comprises generating a set of local vectors between locations of the identified retroreflectors.

In some embodiments, a method for tracking a patient in a frame of reference of a head-mounted display, the patient having a plurality of fiducial objects individually attached thereto, each of the fiducial objects comprising a radiopaque element therein and a retroreflector thereon. the method comprising accessing a fluoroscopic image of the fiducial objects, identifying. in each of the fiducial objects, respective locations of the radiopaque element therein, in response to the identified respective locations, defining a first shape of the attached fiducial objects and formulating a vector between a selected point of the fiducial objects and the patient, so as to register the fiducial objects with the patient, acquiring an optical image of the fiducial objects in response to optical radiation transmitted from the head mounted display, and identifying the retroreflectors in the optical image, formulating a second shape of the attached fiducial objects in response to the identified retroreflectors, and when the second shape corresponds to the first shape, using the identified retroreflectors to track the patient in the frame of reference of the head mounted display.

The embodiments will be more fully understood from the following detailed description thereof, taken together with the drawings.

### BRIEF DESCRIPTION OF THE DRAWINGS

Non-limiting features of some embodiments are set forth with particularity in the claims that follow. The following drawings are for illustrative purposes only and show non-limiting embodiments. Features from different figures may be combined in several embodiments.

FIG. 1 is a schematic illustration of an image-based surgical navigation system for facilitating a medical procedure on a subject, in accordance with an embodiment of the present disclosure;

FIG. 2 is a schematic illustration of a technique for recording landmark locations and validating a registration, in accordance with an embodiment of the present disclosure;

FIGS. 3A and 3B are schematic illustrations of output on a display, in accordance with an embodiment of the present disclosure;

FIG. 4 is a flow diagram for a method for validating a registration, in accordance with another embodiment of the present disclosure;

FIGS. 5A and 5B are illustrations of example screen shots displaying recorded landmarks, in accordance with an additional embodiment of the present disclosure;

FIG. 6A is a schematic illustration of a registration phase of a medical procedure using a set of fiducial landmarks, in accordance with an embodiment of the present disclosure;

FIG. 6B is a schematic illustration of the medical procedure of FIG. 6A using the set of fiducial landmarks, in accordance with an embodiment of the present disclosure;

FIGS. 7A, 7B, and 7C illustrate views of fiducial landmarks, in accordance with an embodiment of the present disclosure;

FIGS. 8A and 8B illustrate views of a fiducial marker and a fiducial object, in accordance with an embodiment of the present disclosure;

FIG. 9 is a schematic illustration showing a head-mounted display, in accordance with an embodiment of the disclosure;

FIG. 10 is a schematic illustration showing a head-mounted display, in accordance with an embodiment of the disclosure; and

FIG. 11 is a flowchart of steps performed in implementing a registration/tracking algorithm, according to an embodiment of the present disclosure.

### DETAILED DESCRIPTION

### Overview

In some surgical or other interventional or diagnostic procedures on a subject, a physician or other clinical professional may view the subject through a near-eye display on which the physician's view, which is tracked by a tracking system, is augmented with an image of the subject's internal anatomy (e.g., a portion of the patient's spine during a minimally invasive surgical procedure). In some implementations, a virtual image of a surgical or other interventional or diagnostic tool, which is tracked by the tracking system, is displayed at the portion of the image corresponding to the current location of the tool.

In such procedures, a fiducial or a fiducial marker coupled to the subject's body may be used to compute a transformation for registering the patient anatomy or image of the anatomy with the fiducial and/or coordinate system of the tracking system. However, if the fiducial moves with respect to the subject or if the location of the fiducial relative to the location of the subject anatomy changes subsequently to the registration, it may be necessary to re-register or to perform a new registration. The registration procedure may include capturing one or more intraoperative images (e.g., three-dimensional or two-dimensional images) of the patient anatomy and of a fiducial while the fiducial is affixed to the anatomy and recompute a transformation based on the new image(s). The term “transformation” may herein refer to one or more transformations. Such registration methods and fiducials are described, for example, in Applicant's U.S. Pat. No. 9,928,629, U.S.

Patent Application Publication No. 2021/0161614, U.S. Patent Application Publication No. 2022/0142730, U.S. Provisional Patent Application No. 63/389,958 and U.S. Provisional Patent Application No. 63/389,955. The disclosures of all of these patents and applications are incorporated herein by reference.

To address this challenge, embodiments of the present disclosure provide a technique for validating the registration or transformation. This validation technique uses one or more landmarks, comprising stickers for example, which are placed on the subject's skin following the registration and, in some implementations, prior to the procedure. However, the landmarks may also be placed during the procedure.

In some embodiments, each of the landmarks is contacted with the surgical or other interventional or diagnostic tool. In some embodiments, the landmarks may alternatively or additionally be contacted with any other element tracked by the tracking system. By virtue of the tool being tracked by the tracking system, the location of the landmark with respect to the tracking system, which is equivalent to the location of the tip of the tool, is determined or ascertained. By utilizing or applying the registration, the location of the landmark with respect to the image may be computed.

Subsequently, during the procedure, at least some of the landmarks may be contacted again, and the locations of these landmarks with respect to the image may be computed. If at least one of these subsequent locations deviates from the corresponding initial location of the landmark by more than a threshold, the registration or transformation may be deemed-by the user (e.g., a physician or a medical professional), or automatically-to be invalid.

Advantageously, embodiments of the present disclosure further provide techniques for identifying a local landmark shift (e.g., due to a skin shift or any other incident which may cause a single landmark or only a specific group or localized portion of the landmarks to move) but may not necessitate re-registering and recomputing of the transformation.

For example, in response to identifying a deviation for one of the landmarks, the distance between the one landmark and another landmark (or all of the other landmarks) may be computed. If this “pairwise distance” has changed, it is more likely that the skin has shifted at the location of the deviated landmark; otherwise, it is more likely that the registration or transformation is invalid. In some embodiments, the number of deviated landmarks may be ascertained. If the number of deviated landmarks is relatively low (e.g., below a threshold percentage or ratio), a skin shift is more likely; otherwise, it is more likely that the registration or transformation is invalid.

Mounting the fiducial marker on an anchoring device such as a clamp clamping a bone, e.g., a spinous process, or a pin inserted to the iliac, may have disadvantages in some implementations. The clamp is, of necessity, located in the region where the surgeon is operating. Because of its size, the clamp may require that the incision made by the surgeon is larger than would be required without use of a clamp. Because of the clamp's location in some implementations, the clamp may restrict the surgeon's access to the region, and it may also at least partially block the surgeon's field of view of the region. An iliac pin may, in some implementations, restrict the surgery location being limited to the iliac bone or iliac crest and the posterior superior iliac spine. In some implementations, the insertion of both pin and clamp requires more invasive operations to be performed on the patient. Embodiments described herein may advantageously allow for less invasive operations to be performed with self-sealing incisions or smaller incisions.

In accordance with several embodiments of the present disclosure, the above disadvantages are overcome by not using a device anchored to the bone of the patient as an anchoring device for the fiducial marker. Rather, in some embodiments, the registration and tracking elements are incorporated into a plurality of individual fiducial objects that are removably attached to the patient's skin. In one embodiment, each fiducial object is formed as a radiotransparent plate. A first surface of the plate may be coated with an optical retroreflector, and an adhesive layer may be formed on a second surface of the plate. The adhesive of the layer has the property that it may be used to removably attach the plate to the patient's skin. In some embodiments, a radiopaque element such as a bead is incorporated within the plate. The radiopaque element may have any symmetrical shape wherein a centroid may be calculated. Non-limiting shapes for an element include a cylinder, an ellipsoid, and a sphere. Multiple radiopaque elements may also be incorporated within the plate.

In an initial stage of an embodiment of the procedure, a set of the fiducial objects is attached (e.g., in a random pattern) to the skin of the patient. The set of the fiducial objects together act as a fiducial marker. In one embodiment, the fiducial objects (e.g., of the random pattern) surround the site of an incision. In one embodiment, the fiducial objects do not surround the site of the incision, but are located in a localized region of the skin separated spatially from the site of the incision.

The set of the fiducial objects and the patient are then scanned by a medical imaging modality (e.g., fluoroscopic scanning by a fluoroscopic imaging device or scanner) and acquired images from the scan are used to register the set of fiducial objects with the skeleton of the patient. The scan may also enable determination of the pattern (e.g., random pattern) of the set of fiducial objects.

In a subsequent stage of the procedure, after the registration has been performed, and, in some implementations, after an incision is made in the patient, the fiducial objects may be irradiated with optical radiation, such as infra-red light. Optical images of the object retroreflectors may be acquired, and the optical images may be analyzed to identify the pattern (e.g., the random pattern), determined in the initial stage, of the fiducial objects. Once the pattern has been identified, it may be tracked, and, because of the registration, images of the patient and of tools used during the procedure may be correctly aligned one with the other and/or correctly aligned with the actual scene and the patient's actual anatomy when presented to the surgeon or other medical professional performing the procedure. It will be understood that while the tracking may be performed after an incision is made in the patient, it may also be performed before the incision is made.

In some embodiments, rather than the fiducial objects being formed individually, and being used to generate an image pattern (e.g., random image pattern), a group of fiducial objects is fixed, in a preset spatial relationship with respect to each other, to a first surface of a flexible sheet. Each fiducial object may be as described above, except that the second surface of the object plate may have no adhesive layer but may be attached to the first surface of the flexible sheet. The adhesive layer may be formed on a second surface of the flexible sheet.

The flexible sheet, now assumed to be in the form of a “sticker.” may be attached to the patient, for example at the localized region described herein, using the adhesive layer. The registration and tracking may be implemented substantially as described above, but the image processing is simplified since the spatial relationship between the elements is known.

### System Description

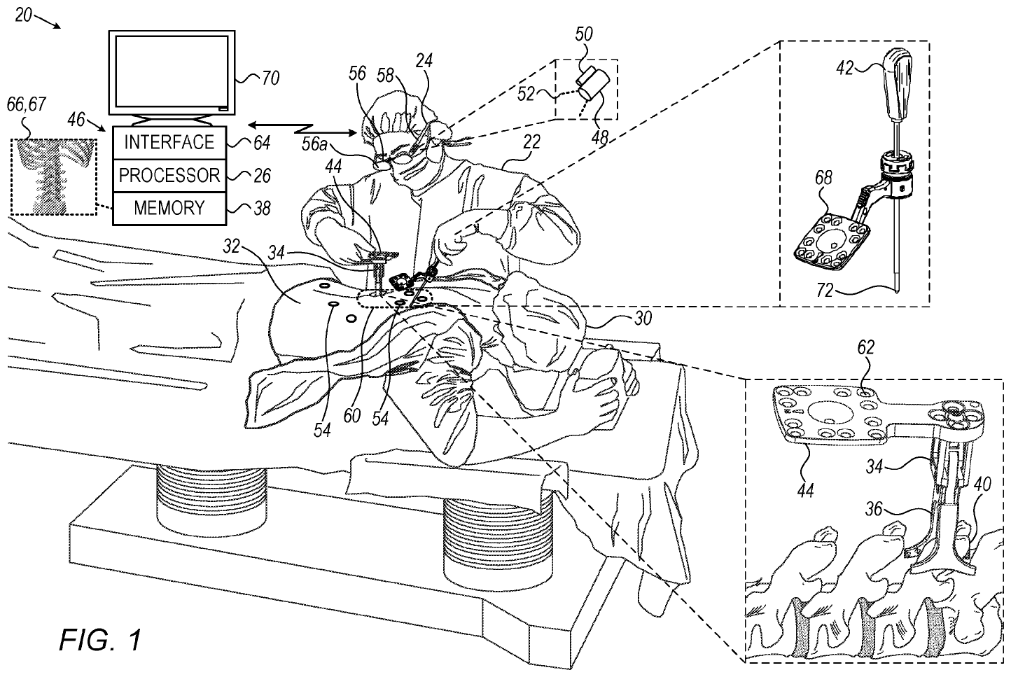

Reference is initially made to FIG. 1, which is a schematic illustration of an image-based surgical navigation system 20 for facilitating a medical procedure on a subject 30, in accordance with some embodiments of the present disclosure. The medical procedure may be a surgical procedure, an interventional procedure, and/or a diagnostic procedure.

By way of example, FIG. 1 depicts a physician or other clinical professional 22 performing a surgical procedure on the back 32 of subject 30. In particular, physician 22 operates on the subject by manipulating a tool 42 in the relevant work area 60, which may include areas above and/or beneath the skin of the subject 30. Embodiments of the present disclosure may be applied to any suitable type of image-guided medical procedure, such as minimally invasive surgeries or open surgeries and may apply to such medical procedures performed on the spine or on any other body portion, such as the shoulder, knee, hip, leg, arm, wrist, skull/brain, chest, heart, and/or abdomen.

System 20 is configured to facilitate the procedure by tracking a fiducial 44, e.g., a patient marker, coupled to the subject 30. In particular, system 20 tracks the location and orientation of fiducial 44, which is indicative of the location and orientation of subject 30.

For example, system 20 may comprise a camera 48, configured to image a field of view (FOV) 52 that includes work area 60. Optionally, system 20 may further comprise a projector or other light source 50, configured to project light within FOV 52 such that the light is reflected back to, and thus sensed by, camera 48. System 20 may further comprise a processor 26, configured to ascertain the location and orientation of fiducial 44 by identifying the fiducial in images acquired by camera 48.

For example, camera 48 may be configured to sense infrared light, and projector 50 may be configured to project infrared light. In some embodiments, camera 48 is configured to sense visible light, such that projector 50 is not required. In some embodiments, system 20 may comprise multiple cameras configured to sense light belonging to different respective portions of the electromagnetic spectrum or may comprise a single camera configured to sense light belonging to different respective portions of the electromagnetic spectrum.

In some embodiments, fiducial 44, which may be referred to as a “fiducial marker,” comprises a plurality of optical elements 62, comprising respective retroreflectors, for example. Optical elements 62 may be arranged in any suitable two-or three-dimensional pattern with no rotational axis of symmetry and no mirror plane of symmetry, such that the positions of optical elements 62 in any image indicate the location and orientation of fiducial 44. Thus, the processor 26 may ascertain the location and orientation of fiducial 44 by identifying the optical elements 62 using image-processing techniques. Such embodiments are described, for example, in Applicant's U.S. Pat. Nos. 10,939,977 and 11,389,252, whose respective disclosures are incorporated herein by reference.

In some embodiments, fiducial 44 is tracked electromagnetically. For example, the fiducial 44 may comprise one or more coils, and system 20 may comprise a magnetic-field generator configured to induce a signal in the coils. Based on the signal, processor 26 may ascertain the location and orientation of the fiducial. Alternatively or additionally, the fiducial 44 may be tracked using any other suitable technique, such as inertial tracking, acoustic tracking, wireless tracking or any other tracking technology or a combination of such technologies.

Fiducial 44 may be coupled to an anchoring device 34 coupled to the subject's spine. For example, anchoring device 34 may comprise a clamp 36 or a pin. Subsequently to inserting the anchoring device 34, e.g., clamp 36, through an incision in back 32, the physician 22 may adjust anchoring device 34 such that opposing jaws of clamp 36 grip at least one spinous process 40 of the subject 30. Alternatively, an anchoring device 34 may comprise a pin, which may be inserted into the iliac bone or other bone of the subject 30.

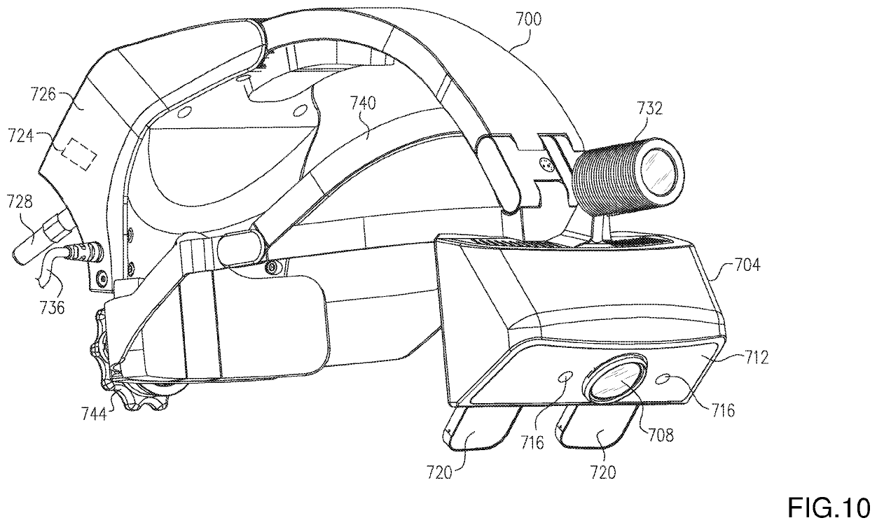

In some embodiments, camera 48 is coupled to an augmented reality assembly or head-mounted display 24 comprising a near-eye display 56. Augmented reality assembly 24 may be worn by the physician 22 such that the physician 22 views work area 60 through near-eye display 56. For example, augmented reality assembly 24 may comprise an eyeglass frame 58, and near-eye display 56 may comprise a pair of display modules 56a (e.g., lenses or loupes) gripped by frame 58 such that, when eyeglass frame 58 is worn by the physician, the physician's line of sight passes through display modules 56a. In some embodiments, augmented reality assembly 24 may comprise a helmet or a headset, and display modules 56a may be mounted to the helmet or headset. e.g., as described with reference to FIG. 10. Alternatively, augmented reality assembly 24 may have any other suitable form (e.g., a visor or portal positioned or mounted between the physician 22 and the subject 30).

In some embodiments, processor 26 is contained in a console 46, which may be positioned near subject 30 during the procedure. In some embodiments, processor 26 may be coupled to (e.g., disposed within a portion of) augmented reality assembly 24. In some embodiments, processor 26 includes two or more processors. In some embodiments, one or more processors are contained in console 46 and one or more couples are coupled to (e.g., disposed within) augmented reality assembly 24. The one or more processors in various locations may share processing tasks (e.g., parallel processing) in some instances and the one or more processors at a single location may perform all of the processing tasks in some instances.

System 20 may further comprise a wired or wireless communication interface 64, via which the processor 26 communicates with other components of the system 20. Thus, for example, the processor 26 may receive images acquired by camera 48 via communication interface 64 and another communication interface (not shown in FIG. 1) coupled to augmented reality assembly 24. For embodiments in which processor 26 is contained in console 46, communication interface 64 may also be contained in the console.

System 20 further comprises a volatile or non-volatile memory 38 configured to store a representation 66 of internal anatomy of subject 30, such as the subject's lumbar spine, lumbosacral spine, cervical spine, and/or thoracic spine. Representation 66 may include, for example, one or more two-dimensional or three-dimensional images 67, which may be acquired using any suitable imaging modality, such as computerized tomography, magnetic resonance imaging, ultrasound imaging, or fluoroscopy. In some embodiments, representation 66 may include a three-dimensional model of the subject's internal anatomy, which may be constructed from images 67. Processor 26 is configured to read or otherwise access representation 66 from memory 38. In some embodiments, processor 26 is configured to generate representation 66.

In some embodiments, system 20 further comprises a display 70, configured to display output from processor 26 as further described below. In some embodiments, at the start of the procedure, the system 20 (e.g., via execution of program instructions stored in memory 38 by processor 26) obtains a transformation for registering representation 66, e.g., with the coordinate system with respect to which fiducial 44 is tracked.

For example, a registration marker, which is visible to the imaging modality used to acquire images 67, may be coupled to anchoring device 34 during the acquisition of images 67. Subsequently, the registration marker may be replaced with fiducial 44. The system 20 (e.g., via execution of program instructions stored in memory 38 by processor 26) may then compute the transformation by correlating between the position and orientation of the registration marker, as shown in images 67, and the position and orientation of the fiducial (as shown, for example, in an image acquired by camera 48). In some embodiments, the position and orientation of the registration marker with respect to the position and orientation of the fiducial 44 may be predefined. Accordingly, system 20 (e.g., via execution of program instructions stored in memory 38 by processor 26) may read the predefined transformation from memory 38 or receive the transformation via communication interface 64. Such a registration marker is disclosed, for example, in U.S. Patent Application Publication No. 2022/0142730, incorporated by reference hereinabove. In some embodiments, the registration marker may include retroflecting elements detectable by the tracking system and may be positioned in a position selected by the physician 22. The transformation between the position and orientation of the registration marker and fiducial 44 may be then calculated based on an image of both the registration marker and fiducial 44 captured by the tracking system. In some embodiments, such a registration marker may be coupled to the patient skin (e.g., adhered). Such a registration marker is disclosed, for example, in U.S. Patent Application Publication No. 2021/0161614, incorporated by reference hereinabove. Once registration is performed, the registration marker may be removed.

Subsequently to obtaining the transformation, the system 20 (e.g., via execution of program instructions stored in memory 38 by processor 26) uses the transformation to facilitate the procedure by displaying representation 66 in registration with the coordinate system in which fiducial 44 is tracked.

For example, for embodiments in which camera 48 is coupled to augmented reality assembly 24, the system 20 (e.g., via execution of program instructions stored in memory 38 by processor 26) may use the transformation to display representation 66 on near-eye display 56 so as to augment the view of the physician 22. Thus, the physician 22 may see, in a minimally invasive procedure, for example, a particular portion of anatomy (such as a portion of the subject's lumbar spine or lumbosacral spine) as the physician 22 looks at the skin covering that portion. In some embodiments, FOV 52 may have a fixed relationship with the physician's field of view. In some embodiments, FOV 52 may be aligned by the physician 22, e.g., vertically, to include the physician's field of view.

In some embodiments, another fiducial 68 may be coupled to tool 42, and the system 20 (e.g., via execution of program instructions stored in memory 38 by processor 26) may track fiducial 68 as described above for fiducial 44. The system 20 (e.g., via execution of program instructions stored in memory 38 by processor 26) may thus track the position and orientation of a distal tip 72 of the tool 42, given the known displacement between distal tip 72 and fiducial 68.

In such embodiments, the system 20 (e.g., via execution of program instructions stored in memory 38 by processor 26), using the transformation, may display representation 66 with an overlaid virtual representation of the tool 42 at the portion of the representation corresponding to the location of the tool 42. Alternatively to a virtual representation of the tool 42 itself, the system 20 (e.g., via execution of program instructions stored in memory 38 by processor 26) may display a virtual tool-tip marker, such as a circle or another symbol or graphic, indicating the location of distal tip 72. Thus, the physician 22 may visually ascertain the location of distal tip 72 with respect to the subject's internal anatomy, even if the distal tip 72 is concealed by skin of the subject 30.

For example, representation 66, optionally with the overlaid virtual representation of the tool 42 or virtual tool-tip marker, may be displayed on near-eye display 56 and/or display 70.

During the procedure, fiducial 44 may move with respect to the subject's anatomy, for example, due to pressure applied to the patient anatomy (e.g., spine) during the procedure. For example, anchoring device 34 may move with respect to the subject's spine, or the spine itself or the bone to which anchoring device 34 is affixed to may move with respect to the rest of the subject's anatomy or body. In such an instance, the registration may become invalid.

Hence, system 20 (e.g., via execution of program instructions stored in memory 38 by processor 26) is further configured to validate the registration or transformation during the procedure, for example as described in detail below with reference to the subsequent figures. To facilitate this validation, one or more (e.g., two or more) landmarks 54 are placed on the skin of the subject 30, e.g., within FOV 52 near work area 60. (Landmarks 54 may be placed before or after the transformation is obtained.) Each landmark 54 may comprise, for example, a symbol (such as a circle or an “x”) drawn with a sterile marker or pen, or may be in the form of a sticker stuck (e.g., adhered) to the skin. For three or more landmarks 54, the landmarks 54 may not all be collinear.

In some embodiments, the landmarks 54 are serialized. For example, one landmark may comprise a “1” drawn on or adhered to the skin, another landmark may comprise a “2,” etc.

In some embodiments, the distance from fiducial 44 to each of landmarks 54 may be at least 10 cm. In some embodiments, the distance from fiducial 44 to one or more of landmarks 54 may be less than 10 cm. (In accordance with several embodiments, larger distances from fiducial 44 provide for a more effective validation of the transformation, relative to smaller distances.) In some embodiments, the distance between each pair of landmarks 54 is at least between three to five cm. In some embodiments, the distance between each pair of landmarks 54 is at least between two to six cm. In some embodiments, the distance between each pair of landmarks 54 is at least between five to ten cm, between four to six cm, between two to seven cm, between one to four cm, between three to six cm, between four to eight cm, between five to ten cm, overlapping ranges thereof, or any value within the above-recited ranges.

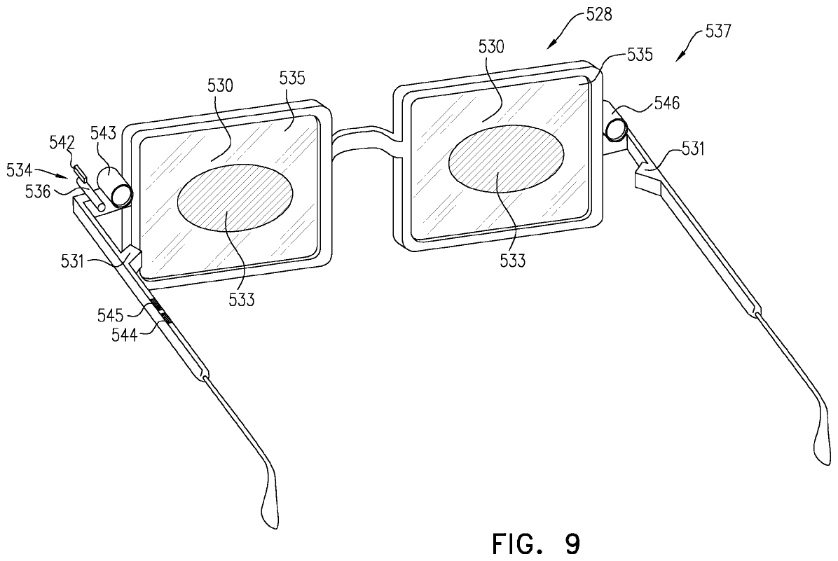

Reference is now made to FIGS. 9 and 10, which show schematic illustrations of different configurations for head-mounted display (HMD) 24. FIG. 9 is a schematic pictorial illustration showing details of head-mounted display unit 537, in accordance with an embodiment of the disclosure. Head-mounted display unit 537 is in the form or substantially in the form of glasses. Head-mounted display unit 537 includes see-through displays 530, for example as described in U.S. Pat. No. 9,928,629 incorporated by reference hereinabove and/or PCT International Publication No. WO 2022/053923, the disclosure of which is incorporated herein by reference. The see-through displays 530 may comprise optical see-through displays, video see-through displays, or a hybrid combination of both. The see-through displays 530 may together comprise a stereoscopic display. Displays 530 may include, for example, an optical combiner, a waveguide, or a visor. Displays 520 may be controlled by a processor 545 and/or by a processor external to HMD 528 to display images to a surgeon such as surgeon 22, who is wearing the HMD 537. The images may be projected onto an overlay area 533 of displays 530 by projectors 531, e.g., in alignment with the anatomy of the body of a patient such as patient 30, which is visible to the surgeon through displays 530. Processor 545 may include more than one processor.

To align images with the patient's anatomy, one or more cameras 536, which may be similar to camera 48 of FIG. 1, capture respective images of a field of view (FOV), which includes fiducials such as fiducial 44 and/or 68. In some embodiments, camera 536 may be an infra-red camera. HMD 537 may then include an infra-red light projector or other light source 542 similar to projector or other light source 50 of FIG. 1. Processor 545 may process the images of one or more of the fiducials to determine and register the location and orientation of display unit 535 with respect to or with the patient's body. In some embodiments, HMD 537 may include one or more (e.g., two or more) additional cameras 543 to provide additional functionality to HMD 537. Cameras 543 may be, for example, video cameras or visible light cameras. In some embodiments, cameras 543 (e.g., a left camera and a right camera) may provide a stereoscopic display of at least a portion of the field of view (e.g., FOV 52) of the surgeon (e.g., physician 22) and may also provide a digital stereoscopic magnification of a portion of the field of view, as disclosed, for example, in Applicant's International Patent Application No. PCT/IB2022/057735, the disclosure of which is hereby incorporated by reference. When displaying a magnified portion of the FOV (e.g., FOV 52), and/or when overlaying and optionally aligning a magnified image portion of the FOV (e.g., FOV 52) with the scene viewed by the surgeon through displays 530, it may be especially important to have the display, camera 536 and/or the image accurately registered with the patient anatomy. In some embodiments, HMD 537 may include an inertial-measurement unit 544 disposed on the HMD 537 to sense movement of the user's head. The inertial-measurement unit 544 may aid in determining the portion of the FOV the physician is focusing on. The camera 536, projector 542, cameras 543, processor 545, and/or inertial measurement unit 544 may form the tracking system.)

FIG. 10 is a schematic pictorial illustration showing details of HMD 700, according to an embodiment of the disclosure. HMD 700 may be worn by a surgeon such as surgeon 22 and may be used in place of HMD 537 of FIG. 9 or HMD 24 of FIG. 1. HMD unit 700 includes an optics housing 704 which incorporates a camera 708, and in the specific embodiment shown, an infra-red camera. Thus, housing 704 also comprises an infra-red transparent window 712, and within the housing, e.g., behind the window 712, are mounted one or more, e.g., two, infrared projectors 716. Mounted on housing 704 are a pair of augmented reality displays 720, which may allow the surgeon to view entities, such as part or all of a patient such as patient 30 through the displays 720, and which are also configured to present to the surgeon images or any other information.

HMD 700 may include a processor 724, mounted in a processor housing 726, which may operate elements of the HMD unit 700. An antenna 728, may be used for communication, e.g., with workstation 46 of FIG. 1. The processor 724, the camera 708, and/or the projectors 716 may form the tracking system.

Optionally, a flashlight 732 may be mounted on the front of HMD 700. The flashlight 732 may project visible spectrum light onto objects so that the surgeon may be able to see more clearly objects through displays 720. Elements of the HMD 700 may be powered by a battery (not shown in the figure) which supplies power to the elements via a battery cable input 736.

HMD 700 may be held in place on the head of a surgeon by a head strap 740, and the surgeon may adjust the head strap by an adjustment knob 744.

It should be appreciated that elements and functionalities described with respect to each of HMD 24, HMD 528 and HMD 700, may apply to each of the other HMDs, mutatis mutandis.

In general, processor 26, processor 545 and/or processor 724 may be embodied as a single processor, or as a cooperatively networked or clustered set of processors. The functionality of these processors may be implemented solely in hardware, e.g., using one or more fixed-function or general-purpose integrated circuits, Application-Specific Integrated Circuits (ASICs), and/or Field-Programmable Gate Arrays (FPGAs). Alternatively, this functionality may be implemented at least partly in software. For example, any one of the processors may be embodied as a programmed processor comprising, for example, a central processing unit (CPU) and/or a Graphics Processing Unit (GPU). Program code, including software programs, and/or data may be loaded for execution and processing by the CPU and/or GPU. The program code and/or data may be downloaded to the processor(s) in electronic form, over a network, for example. In some embodiments, the program code and/or data may be provided and/or stored on non-transitory tangible media, such as magnetic, optical, or electronic memory. Such program code and/or data, when provided to the processor(s), produce a machine or special-purpose computer, configured to perform the tasks described herein. The processors may perform tasks in parallel or separately in various configurations.

### Recording Landmarks and Validating the Registration

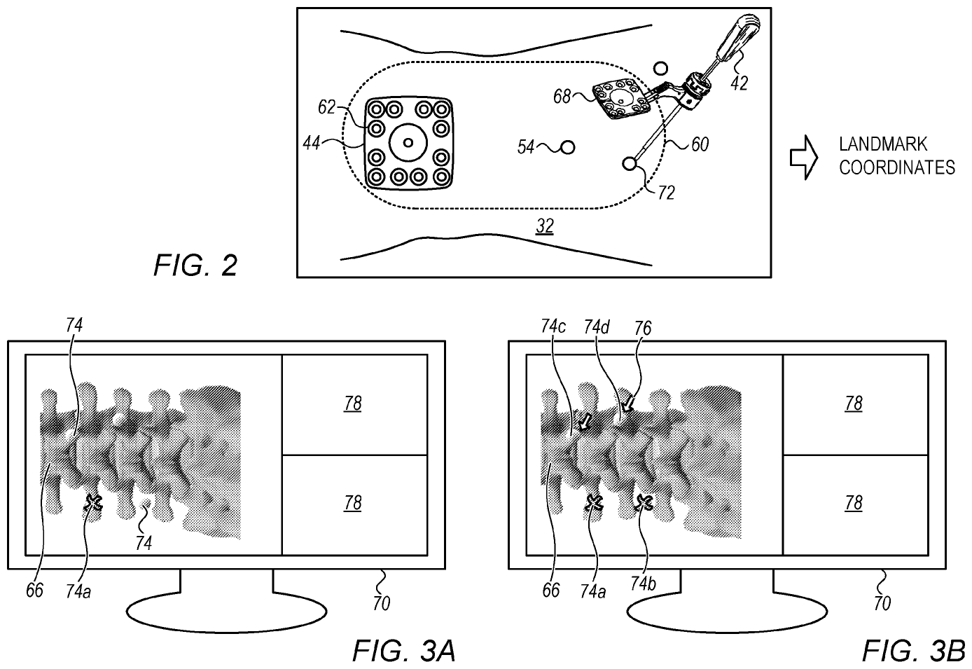

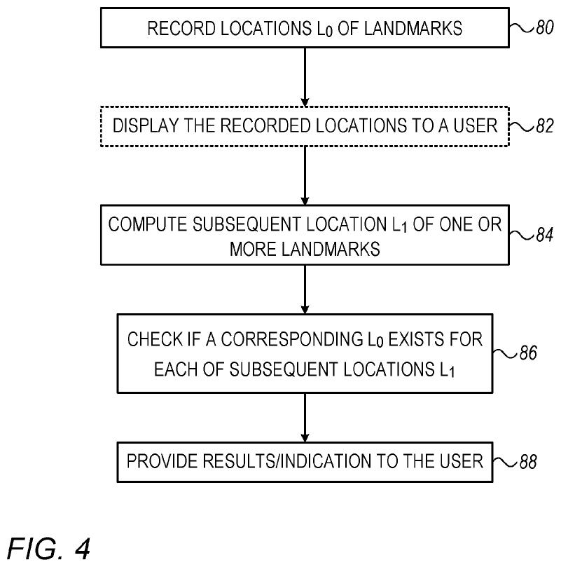

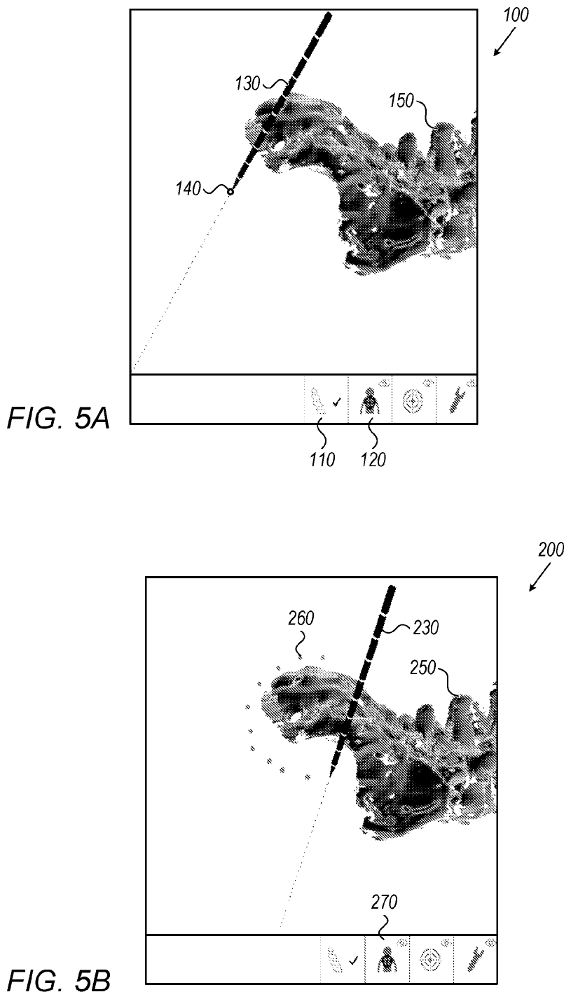

Reference is now made to FIGS. 2, 3A, 3B and 4. FIG. 2 is a schematic illustration of a technique for defining and recording initial landmark locations and validating a registration or transformation, in accordance with some embodiments of the present disclosure. FIGS. 3A-B are schematic illustrations of output on a display, displaying the defined landmarks and validation check results and/or indications in accordance with some embodiments of the present disclosure. FIG. 4 is a flow diagram for a method for validating a registration, in accordance with some embodiments of the present disclosure. The method of FIG. 4 may be executed by processor 26 (FIG. 1) and/or the processor of an HMD such as processor 545 or 724.

Reference is now made to FIG. 2, which depicts a portion of back 32 of the subject 30. At the start of the procedure, the physician (or another user) 22 may determine and/or generate landmarks 54, e.g., by marking or drawing them on selected locations on the skin of the subject's back 32 with a sterile marker or by coupling the landmarks to selected locations of the skin of the subject 30 (e.g., by adhering landmarks in the form of stickers). In some embodiments, the system 20 (e.g., via execution by processor(s)) may compute and suggest locations for the landmarks 54 with respect to a presentation of the anatomy of the subject 30, such as presentation 66 as shown in FIGS. 3A and 3B, e.g., by displaying suggested locations on the presentation on the HMD display and/or the workstation display.

Referring now to FIG. 4, at a step 80, once the landmarks 54 are defined, the locations of the landmarks 54 are recorded by the processor or the system, e.g., processor 26 and system 20 of FIG. 1. The user may contact each of landmarks 54 with a predetermined portion of tool 42 (e.g., distal tip 72), which, as described above with reference to FIG. 1, is coupled to fiducial 68. In some embodiments, another tool or device coupled to a tracked fiducial, rather than tool 42, may be used to contact the landmarks 54. (Such a tool may be dedicated for this purpose, i.e., the tool or device may not be used to operate on the subject or perform any other function.)

While each of the landmarks 54 is contacted, the physician (or another user) 22 instructs the system or processor (e.g., system 20 or processor 26 of FIG. 1) to record the coordinates of the landmark 54. This instruction may be performed using any suitable input device such as a foot pedal, a mouse, a keyboard, a touch pad, a voice command or a touch screen, e.g., belonging to display 70 (FIG. 1) or a handheld computing device or HMD. The instruction may be provided via communication interface 64.

In response to the instruction, and given the known displacement between fiducial 68 and the predetermined portion of the tool that contacts the landmarks, the system 20 (e.g., via processor 26, 545, 724) computes the respective initial coordinates of the landmarks, e.g., in the tracking coordinate system. In particular, the system 20 (e.g., processor 26) computes {B₀ⁱ} for i=1 . . . N, where B₀ⁱ, a three-dimensional vector, is the initial location of the i^{th} landmark in the tracking coordinate system and N is the number of landmarks.

Subsequently, by applying the transformation obtained as described above with reference to FIG. 1. the system 20 (e.g., via processor 26, 545, 724) computes the initial coordinates of the landmarks relative to representation 66 (FIG. 1). In particular, the system 20 (e.g., via processor 26, 545, 724) computes a location L₀ for each landmark i: {L₀ⁱ} for i=1 . . . N, where L₀ⁱ, a three-dimensional vector, is the initial location of the i^{th} landmark in the coordinate system of representation 66, by transforming each B₀ⁱ per the transformation. In other words, using the notation T( ) to designate the transformation operation, the system 20 (e.g., via processor 26, 545, 724) calculates each L₀ⁱ as T(B₀ⁱ) or T⁻¹(B₀ⁱ), depending on whether T transforms from or to the tracking coordinate system.

During the procedure, the transformation may be used to display representation 66 in registration with the tracking coordinate system. For example, as described above with reference to FIG. 1, representation 66 may be displayed on near-eye display 56 and/or display 70. In some embodiments, representation 66 may be displayed on near-eye display 56 and/or display 70 in registration with a virtual representation of tool 42. Alternatively or additionally to the virtual representation of the tool, the system 20 (e.g., via processor 26, 545, 724) may display respective virtual representations of the landmarks 74 at the initial coordinates, as shown in FIGS. 3A-B. Each of these virtual representations may include a sphere or any other suitable symbol (e.g., X-shaped symbol, ellipsoid, cross-hair symbol).

It should be appreciated that in order to provide the aligned display as herein disclosed (e.g., anatomy representation and tool representation alignment and/or anatomy representation and actual patient anatomy as viewed by the user through the HMD see-through display alignment), representation 66, representation of the tool and/or any other images and the tracking system data or measurements should, in accordance with several embodiments, be represented in a predefined common coordinate system (e.g., by registration or transformation). Such a common coordinate system may be, for example, a coordinate system defined by the fiducial, e.g., fiducial 44 of FIG. 1 or any other coordinate system. The aligned images and/or representations in the common coordinate system may be then, for example, transformed to the display coordinate system for purposes of display.

At an optional step 82, the system 20 (e.g., via processor 26, 545, 724) displays the recorded landmarks overlaid on an image or a representation of the patient anatomy (e.g., a region of interest (ROI) of the patient anatomy) based on tracking or detection of the defined locations of the landmarks, e.g., by tracking the tip of the tool, and the registration. Such a display, for example, of landmarks 74 is shown in FIGS. 3A and 3B.

Subsequently to the determination and recording of landmarks 54, a validation procedure for validating the transformation may be performed. The validation procedure may be performed in response to an instruction from the physician or may be performed automatically. Such an instruction may be performed using any suitable input device, such as the input devices indicated above. In some embodiments, the user may be advised, e.g., via a displayed message, to perform the validation procedure after a predetermined amount of time has transpired from the computation of the initial landmark coordinates or prior to or following a specific operation, e.g., screw(s) placement or bone cut.

During the validation procedure, tool 42 (or another tool or device, e.g., a dedicated pointing device) is again used to contact at one or more of landmarks 54. In response to the tracking of fiducial 68 and by applying the transformation as described above, the system 20 (e.g., via processor 26, 545, 724) computes, as a step 84, a subsequent location L₁ for each of the validated landmarks k: {L₁^{k}}, which includes subsequent coordinates of landmarks, for k=1 . . . M, where M≤N is the number of contacted landmarks. In some embodiments, for each of the contacted landmarks k, the physician or another user indicates to the system 20 (e.g., processor, 545, 724 via interface 64 or other input device, such as input window 78) which of the defined landmarks i is being contacted, e.g., by selecting the virtual representation of the landmark on display 70 (FIG. 1).

Accordingly, during the procedure, an additional or a second subsequent validation check may be performed, following the previous or first validation check. The system 20 (e.g., via processor 26, 545, 724) may then compute {L₂^{j}}, which includes additional subsequent coordinates of landmarks, for j=1 . . . P, where P≤N is the number of contacted landmarks at the second validation check. The user may initiate one or more such registration validations during the medical procedure, as required or as desired by the user (e.g., a physician or another medical professional).

In some embodiments, the number of landmarks defined N, may be changed by the user, e.g., via interface 64, during the procedure, e.g., increased by defining additional landmarks or decreased by deleting previously defined landmarks. Accordingly, one or more landmarks may be replaced during the procedure, e.g., by deleting a previously defined landmark and defining a new one instead and such that the number N of defined landmarks will stay the same. In some embodiments, the number of landmarks to be determined is limited. For example, a maximum number of landmarks which may be defined is predetermined, e.g., five landmarks, ten, fifteen or any other number in between or higher than fifteen. Limiting of the number of landmarks which may be defined may, for example, simplify the validation or assist in assuring that a minimum distance between landmarks will be kept.

Next, at a step 86, the system (e.g., via processor 26, 545, 724) may check if a corresponding L₀, i.e., a corresponding initial location, exists for each of the validated landmarks having subsequent computed locations L₁.

In some embodiments, the system 20 (e.g., via processor 26, 545, 724) computes the distance between at least one of the subsequent coordinates and each of the defined landmark initial coordinates. In other words, for at least one value of k, the system 20 (e.g., processor 26, 545, 724) computes the distance ∥L₁^{k}−L₀ⁱ∥ for each i. (Throughout the present description, the notation ∥v∥ indicates the magnitude of a vector v, which may be calculated as any suitable norm, such as the L1-norm or the L2-norm, of the vector.) A threshold, e.g., a radius, R may be predefined to determine an acceptable error measurement and/or acceptable registration deviation. Thus, if the distance between a validated landmark location and an initial location of a recorded landmark ∥v∥≤R, then the system 20 (e.g., via processor 26, 545, 724) may determine that a corresponding initial location exists and identify the validated landmark as corresponding to the identified recorded landmark. In some embodiments, the system 20 (e.g., via processor 26, 545, 724) may stop the distances calculations once such a corresponding recorded landmark was identified. In some embodiments, the system 20 (e.g., via processor 26, 545, 724) may keep computing the distances for a validated landmark until such distances are received for each recorded landmark. If two or more recorded landmarks were found to be distant from the validated landmark within the predefined threshold R, then the one which is the closest may be defined as the corresponding recorded landmark. In some embodiments, the landmarks may be defined as spheres, e.g., as shown in FIGS. 3A-B, having a radius R. The above assumes that the defined landmarks are placed on the patient such that they are generally sufficiently spaced from each other. Accordingly, it may be desired in some implementations to place each landmark on the patient body at a distance greater than R. In some embodiments, the user may be guided accordingly (e.g., via display of a textual message and/or graphical symbols and/or playing of an audio message).

In some embodiments, for each of the landmarks contacted during the validation, the physician or another user indicates to the system 20 (e.g., processor 26, 545, 724 via interface 64 or input window 78) which of the defined landmarks is being contacted, e.g., by selecting the virtual representation of the landmark on a display, e.g., display 70 (FIG. 1). Thus, the system 20 (e.g., via processor 26, 545, 724) may identify the corresponding initial coordinates L₀ⁱ for each of the subsequent coordinates and check the distance between a validated landmark L₁^{k} and the initial coordinates of its corresponding defined landmark indicated by the user L₀ⁱ. In some embodiments, each landmark generated by the user may have a unique characteristic, e.g., a number, a name, a sign, or a color, uniquely identifying the landmark. When recording the defined landmarks, the system 20 (e.g., via processor 26, 545, 724) may also record or store the unique characteristic for each landmark and in association with the landmark. Each time the user validates a landmark, the user may provide the unique characteristic of the landmark, e.g., via a user interface (e.g., interface 64 or input window 78).

At a step 88, the system 20*e.g., via processor 26, 545, 724) may generate an output in response to the check performed at step 86 as a result or indication to the user. The output may include, for example, a visual output on display 70 and/or near-eye display 56, 535 or 720. For example, for embodiments in which the system 20 (e.g., via processor 26, 545, 724) displays (or generates for display) virtual representations of the landmarks at the initial coordinates, the system 20 (e.g., via processor 26, 545, 724) may modify at least some of the virtual representations, e.g., as described below with reference to FIGS. 3A-B. In some embodiments, the output may include audio output via a speaker.

Reference is now made to FIGS. 3A-B, which show an example representation 66—namely, a three-dimensional model of a portion of a spine—displayed on display 70. Virtual representations 74 of the landmarks (e.g., landmarks 54) are displayed in registration with the representation 33 (e.g., 3D spine model). Also displayed are several input/output windows 78, which may include input interfaces for receiving input, and/or output interfaces for the display of visual outputs. As an example of an input interface, a window 78 may include clickable icons for instructing the system 20 (e.g., processor 26, 545, 724) to add or remove landmarks, to begin or end a validation procedure, and/or to show or hide virtual representations 74.

In some embodiments, the system 20 (e.g., via processor 26, 545, 724) compares each of the measured distances to the predefined threshold R. and generates the output in response thereto. In general, if a distance exceeds the threshold, it is likely that either the landmark has locally shifted, e.g., due to a local skin shift, or that the registration may have become invalid. Hence, as further described below, the output may explicitly or implicitly indicate that one of these two scenarios applies, by modifying virtual representations 74 and/or displaying or otherwise generating appropriate messages or alerts in windows 78 or anywhere else on display 70 (and/or on the near-eye display).

For example, for each of the landmarks (e.g., landmarks 54), if the distance between a validated landmark and a corresponding recorded landmark exceeds the threshold, the virtual representation 74 of the recorded landmark may be modified in one way; otherwise, the virtual representation 74 may be modified in a different way. Alternatively, the virtual representation may be modified only if the threshold is exceeded, or only if the threshold is not exceeded. For example, if a recorded landmark is identified as corresponding to a validated landmark (e.g., a landmark on the patient body which is in a validation process), the color of the corresponding recorded landmark may change.

Each of the aforementioned modifications may include, for example, a change in color, a change in size, and/or a change in form, such as a change in shape and/or the addition of a symbol or caption over and/or adjacent to the current symbol that represents the landmark. For example, as shown in FIG. 3A, if a recorded landmark was determined as not having a corresponding landmark, an “x” may be overlaid over the virtual representation 74a of the landmark, e.g., virtual representation 74a may be crossed out. In some embodiments, the virtual representation of a recorded landmark may be assigned one color (e.g., green) if the recorded landmark was determined valid, i.e., as having a corresponding landmark, and another color (e.g., red) if the recorded landmark was found invalid, e.g., determined as not having a corresponding landmark.

In some cases, alternatively or additionally to modifying virtual representations 74, the system 20 (e.g., via processor 26, 545, 724) may generate an output indicating that the recorded landmark(s) which were not validated should not be used for subsequent validation of the transformation. For example, the system 20 (e.g., via processor 26, 545, 724) may display a message in one of windows 78 indicating that the landmarks whose virtual representations 74 are crossed out or colored red are disqualified from use.

In some embodiments, the output may indicate that one or more landmarks (e.g., landmarks 54) may have locally shifted. Such an output may be generated in response to at least one of the recorded landmarks being determined invalid, given that if all of the recorded landmarks are determined invalid, it is more likely that the registration is invalid. Subsequently, the physician may correct the local shift, or at least refrain from contacting the shifted landmarks during a subsequent validation.

In some embodiments, the system 20 (e.g., via processor 26, 545, 724) may select one or more of the recorded landmarks which were determined to be valid, and generate an output indicating that the selected recorded landmarks should be used for subsequent validation of the transformation. In some cases, the selected recorded landmarks may be positioned at a larger distance, one from the other, with respect to the unselected recorded landmarks.

For example, FIG. 3B shows a scenario in which, as in FIG. 3A, the recorded landmark represented by virtual representation 74a was determined as not having a corresponding landmark on the patient body, indicating that this landmark has likely shifted. In this scenario, the system 20 (e.g., via processor 26, 545, 724) may select other landmarks, represented in FIG. 3B by virtual representations 74c and 74d, which are farther from the shifted landmark than is the unselected landmark represented by virtual representation 74b. To indicate that the selected landmarks should be used, the system 20 (e.g., via processor 26, 545, 724) may, for example, display respective arrows 76 pointing to the virtual representations of these landmarks. Symbols or images other than arrows may also be used. The system 20 (e.g., via processor 26, 545, 724) may also indicate that the unselected landmark should not be used, as done for the shifted landmark.

In some embodiments, prior to outputting an indication that a landmark may have locally shifted, the system 20 (e.g., via processor 26, 545, 724) checks whether the “pairwise distance,” referred to below as the PD, between at least one pair of landmarks has changed. In such embodiments, prior to the first validation procedure, the system 20 (e.g., via processor 26, 545, 724) computes respective initial pairwise distances between one or more pairs of the landmarks. In other words, for each pair consisting of the i^{th} and j^{th} landmarks, the system 20 (e.g., via processor 26, 545, 724) computes the initial PD in either one of the coordinate systems. (In other words, the system 20 (e.g., via processor 26, 545, 724) computes ∥L₀ⁱ−L₀^{j}∥ or ∥B₀ⁱ−B₀^{j}∥). Subsequently, in response to a landmark not having a corresponding recorded landmark, the system 20 (e.g., via processor 26, 545, 724) checks whether the PD between this landmark and another landmark has changed, e.g., the system 20 (e.g., via processor 26, 545, 724) computes the subsequent PD between the pair of landmarks and compares the subsequent PD to the initial PD. Subsequently, the system 20 (e.g., via processor 26, 545, 724) may output an indication of a local shift, e.g., a skin shift, in response to the magnitude (i.e., absolute value) of the difference between the subsequent PD and the initial PD exceeding another predefined threshold.

In some cases, alternatively or additionally to modifying virtual representations 74, the system 20 (e.g., via processor 26, 545, 724) may generate an output indicating that the transformation may have become invalid. For example, the system 20 (e.g., via processor 26, 545, 724) may display a warning in one of windows 78. Such an output may be generated, for example, if for at least a predetermined number of landmarks (e.g., one, two, or three) or percentage (e.g., 10%, 20%, 30%, or 50%) or ration (e.g., 1:10, 1:5, 1:3, 1:4, 1:2) of the landmarks were found invalid since no corresponding recorded landmark was identified for these landmarks. In some embodiments, such an output may be generated if at least one landmark was found invalid, but the PD between this landmark and another one or more landmarks or a percentage or ratio of the landmarks has not changed.

On the other hand, if no recorded landmark was found invalid, the system 20 (e.g., via processor 26, 545, 724) may continue using the registration. Subsequently, at least one more validation procedure may be performed, e.g., after a predetermined amount of time has transpired from the previous validation or in response to an instruction from the physician. In some embodiments, the system 20 (e.g., via processor 26, 545, 724) may display the distance between the initial location and subsequent location, e.g., via a displayed ruler. The user may then decide if a difference between an initial and subsequent location of a landmark is significant or not and if this landmark and/or the registration is still valid.

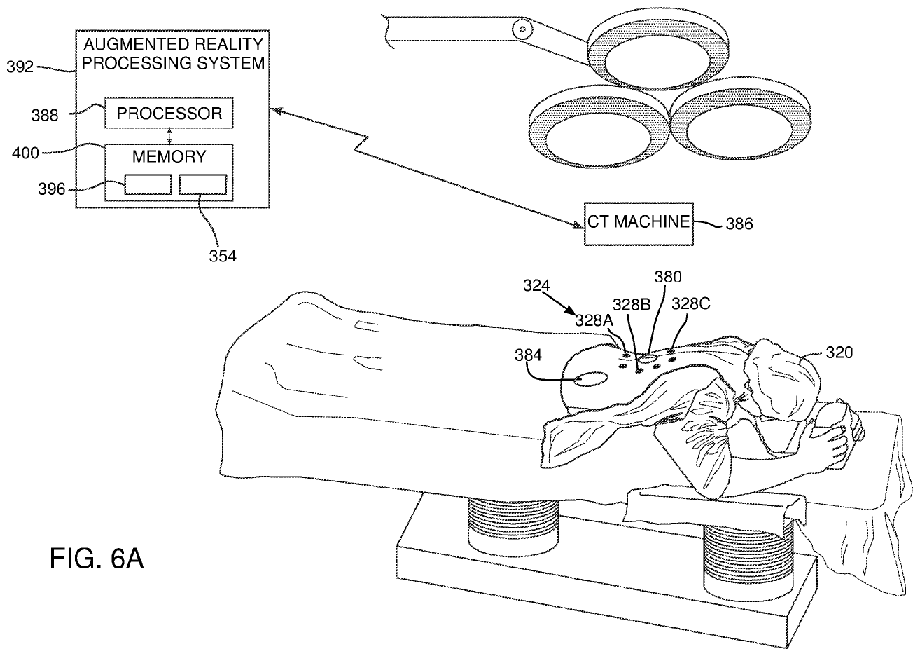

Reference is now made to FIGS. 5A and 5B, which are illustrations of example screen shots 100 and 200, respectively, of a graphical user interface displaying recorded landmarks, in accordance with an embodiment of the present disclosure. A three-dimensional (3D) model 150 representing a portion of a patient spine is displayed on a graphical user interface of the screen or display. A virtual tool image 130 representing a tool used by the physician is also displayed in alignment with 3D model 150. An icon 110 may be checked to allow for recording of landmarks. A tracking system, e.g., camera 48, 536 or 708 of FIGS. 1, 9 and 10 respectively, may track the tool and an icon 120 may be selected for recording a landmark located at the tip of the tool and as displayed as landmark 140. The landmarks may be displayed as dots or circles or other symbols or shapes and may be generated or defined by the system 20 (e.g., by processor 26, 545, 724) as spheres having a predefined radius R. Optionally, the color of the virtual landmark 140 may vary, depending on whether the tip of the tool is touching the recorded location L0 of the landmark on the patient skin.

Reference is now made to FIG. 5B, which shows a display of ten recorded virtual landmarks 260 corresponding to actual landmarks on the patient's skin. In some embodiments, a maximum number X of landmarks or virtual landmarks may be recorded. If an X+1 landmark is recorded, then one of the previously recorded landmarks, e.g., the first recorded landmark, may be automatically deleted. For example, if M=10, then adding the 11th landmark to display 200 would delete one of the ten displayed virtual landmarks. In some embodiments, a user may select a landmark and press a remove landmark button to delete the recorded landmark from the display 200.

In some embodiments, a set of landmarks as disclosed herein above, which may be removably attached to the patient skin is provided. In some embodiments, the landmarks are in the form of stickers. In some embodiments, each landmark includes on a top visible surface of the landmark a unique graphical element, which uniquely identifies each landmark.

In some embodiments, a set of landmarks which may be removably attached to the patient skin is provided while each landmark includes on a top visible surface one or more retroreflective elements. The landmarks may be then used in addition to or instead of the fiducial tracked by the tracking camera, e.g., fiducial 44 of FIG. 1.

If the set of landmarks is used in addition to a fiducial such as fiducial 44, then the registration or transformation may include a performed registration and computed transformation between the set of landmarks and the fiducial. The registration may be performed by capturing an image by the tracking camera (e.g., camera 48) of both the set of markers coupled to the patient skin and the fiducial coupled to the patient anatomy. A transformation may be then computed based on the image. In some embodiments, the transformation may involve the determination of the location and orientation of each landmark of the set of landmarks coupled to the patient skin relative to the fiducial. The registration validation may be performed as described hereinabove, and/or with certain changes as will be described hereinbelow.

If the set of landmarks is used to replace the fiducial, then the registration or transformation may include a performed registration and computed transformation between the set of landmarks and the registration marker. The registration marker may then include one or more retroreflective elements and may be removably coupled to the patient skin as well. Such a registration marker is described, for example, in Applicant's U.S. Patent Application Publication No. 2021-0161614 incorporated by reference herein above. The registration between the set of markers coupled to the patient skin and the registration marker may be performed by capturing an image by the tracking system (e.g., camera 48) of both and computing their relative locations and orientations. The registration validation may be performed by recording the initial layout of the landmarks, as coupled to the patient skin, and checking if the initial layout of the landmarks has changed, e.g., due to a local shift of one or more landmarks. The registration verification may be performed by verifying the distances and orientations of one or more landmarks, each with respect to one or more of other landmarks. In accordance with several embodiments, the registration verification may be similar to the verification described herein above with certain changes.

In some embodiments, the one or more retroreflective elements of each landmark uniquely identify the landmark. In some embodiments, the landmarks may be in the form of stickers. In some embodiments, each landmark may include a unique number of retroflectors. In some embodiments, each landmark may include one or more retroflectors forming a unique shape. The retroflectors may be tracked by a tracking camera. The tracking camera may be head-mounted, such as camera 48, 536, or 708 of FIG. 1, 9 or 10 respectively. In such a case, there may advantageously be no need for touching the landmark with a trackable tool or device in order to define and record a landmark. In case of a head-mounted camera, the user may simply look at the landmarks, which are now trackable by themselves, to record their initial locations and orientations on the patient's body. The recorded landmarks may be displayed on the display (e.g., a display of a workstation such as display 70, and/or a display of an HMD). In some embodiments a user is required to record and/or validate the locations of landmarks located in the tracking system FOV.

An example method or algorithm to be used with the set of retroreflective landmarks is herein disclosed. The method or algorithm may be executed by processor 26 and/or the processor of an HMD (e.g., processors 545, 724). The method begins with the processor obtaining (e.g., computing, determining, accessing, calculating) the transformation for registering representation 66 (FIG. 1) with the tracking coordinate system (e.g., coordinate system of camera 48 536, or 708).

Following the obtaining of the transformation, the initial locations of landmarks 54 (FIG. 1) are recorded by capturing images of the landmark by the tracking camera (e.g., camera 48 536, or 708). In particular, the processor may repeatedly check, whether a landmark appears in the captured images. In some embodiments, the processor may additionally check whether an input instructing landmark recording is received or applies. If yes, the processor may use the transformation to compute the initial locations of the captured landmark(s) relative to representation 66, and may record the locations. Optionally, the processor may also display (or generate for display) the locations (e.g., by displaying a virtual representation of the landmark at the location as shown in FIGS. 3A-B and 5A-B).

Subsequently, the processor may check whether an input indicating that the recording is finished was received. In some embodiments, after a predefined time interval and once no new landmark is captured, the processor may automatically terminate the landmark recording procedure or phase.

According to some embodiments, the processor may repeatedly validate the location of landmarks which appear in images repeatedly captured by the tracking camera. Alternatively, the processor may validate the location of such landmarks only upon user request. The processor may then use the received tracking information and the transformation to compute the subsequent location and orientation of one or more landmarks captured by the tracking camera. Optionally, the processor may display (or generate output for display) the subsequent location. For example, the processor may display, at the subsequent location, a landmark virtual representation having a different shape and/or color from the virtual representations at the initial locations.

Subsequently, the processor may compute the distance between the subsequent and initial landmark locations for each captured landmark. Optionally, the processor may further display a virtual ruler spanning the distance between the initial location and the subsequent location, and/or otherwise indicate the distance on display 70 (FIG. 1) and/or the HMD display, so as to help the physician decide if the deviation in location is significant.

If the set of landmarks is used as a fiducial, initial and subsequent relative distances and optionally orientations of the landmarks, may be imaged, recorded, and compared. The relative distance (and optionally orientations) of pairs of the landmarks may be computed and compared to the initial computed distances (and optionally orientations). For example, if registration and tracking is performed with respect to a center point of the landmark, then orientation may not be tracked and/or considered.

Subsequently, the processor may modify the virtual representation of the landmark responsively to the distance. For example, as described above with reference to FIGS. 3A-B, the processor may modify the color of the virtual representation so as to indicate whether the measured distance between a current location of the landmark and the initial recorded location exceeds a predefined threshold. The predefined threshold may indicate, for example, an acceptable measurement error or registration deviation.

In some embodiments, the processor may check whether there are any landmarks which were not validated. The processor may display an indication to the user, e.g., by indicating on the display landmarks which were not validated. The processor may also cause an audible alert to be generated.

The processor then may check whether the transformation is possibly invalid. For example, the processor may check whether the number of landmarks whose measured distance from the initial location exceeds the threshold is greater than a predefined number. In some embodiments, a set of fiducial landmarks which may be removably attached to the patient skin is provided. Each landmark may include one or more radiopaque elements and one or more retroreflective elements. Thus, the registration and tracking elements are incorporated into the landmarks which will be referred hereinbelow as fiducial landmarks. In one embodiment, each fiducial object is formed as a radiotransparent plate. A first surface of the plate may be coated with an optical retroreflector, and an adhesive layer may be formed on a second surface of the plate. The adhesive of the layer may have the property that it may be used to removably attach the plate to the patient's skin. A radiopaque element such as a bead may be incorporated within the plate. The radiopaque element may have any symmetrical shape wherein a centroid may be calculated. Non-limiting examples of shapes for the radiopaque element include a cylinder, an ellipsoid, and a sphere.

In an initial stage of an example procedure, a set of the fiducial landmarks is attached in a random pattern to the skin of the patient. The set of fiducial landmarks may advantageously act together as a single fiducial marker. In some embodiments, the fiducial landmarks of the random pattern surround the site of an incision. In some embodiments, the fiducial landmarks do not surround the site of the incision, but are located in a localized region of the skin separated spatially from the site of the incision.

In some embodiments, the set of fiducial landmarks and the patient are then scanned by a medical imaging device (e.g., by a Computerized Tomography (CT) device or by a fluoroscope), and acquired images from the scan are used to register the set of fiducial landmarks with the skeleton of the patient. The scan may also enable determination of the random pattern of the set of landmarks. In some embodiments, each fiducial landmark may include one or more uniquely identifying retroreflective elements on a visible top surface of the landmark.

In a subsequent stage of the procedure, after the registration has been performed, and, in some implementations, after an incision is made in the patient, the fiducial landmarks may be irradiated with optical radiation, such as infra-red light, by a tracking camera (e.g., camera 48, 534 or 708). Optical images of the landmarks retroreflectors may be acquired, and the images may be analyzed to identify the random pattern, determined in the initial stage, and/or the uniquely identifying retroflectors of the fiducial landmarks. Once the pattern has been identified, it may be tracked, and because of the registration images of the patient and of tools used during the procedure may be correctly aligned one with the other and/or correctly aligned with the actual scene and the patient's actual anatomy when presented to the surgeon performing the procedure. It will be understood that while the tracking may be performed after an incision is made in the patient, it may also be performed before the incision is made.

The initial locations of the set of fiducial landmarks may be recorded, e.g., during the registration procedure and/or subsequent to the registration. Following that, a validation may be performed by checking the distances between the initial locations and subsequent locations of a landmark, as captured by the tracking camera and with respect to one or more of the other landmarks. A local shift of one or more of the landmarks may be then identified and an alert or some other indication may be output to the user, e.g., via a workstation display, such as display 70, and/or an HMD display, such as display 56a, 530 or 720. In some embodiments, the validation may allow the disqualification of one or more landmarks of the set of landmarks in case they are determined to be invalid. The systems, methods and software products described herein above for landmarks and registration verification may apply, mutatis mutandis, to the disclosed set of fiducial markers and/or to the use of such as will be further detailed below.

In some embodiments, a group of fiducial objects is fixed, in a preset spatial relationship with respect to each other, to a first surface of a flexible sheet. Each fiducial object may be similar to the fiducial landmarks described above, except that the second surface of the object plate may have no adhesive layer, but may be attached to the first surface of the flexible sheet. In some embodiments, the adhesive layer is formed on a second surface of the flexible sheet.

The flexible sheet, now assumed to be in the form of a “sticker,” may be attached to the patient (e.g., at the localized region described herein) using the adhesive layer. The registration and tracking may be implemented substantially as described above, but the image processing may be simplified since the spatial relationship between the elements is known.

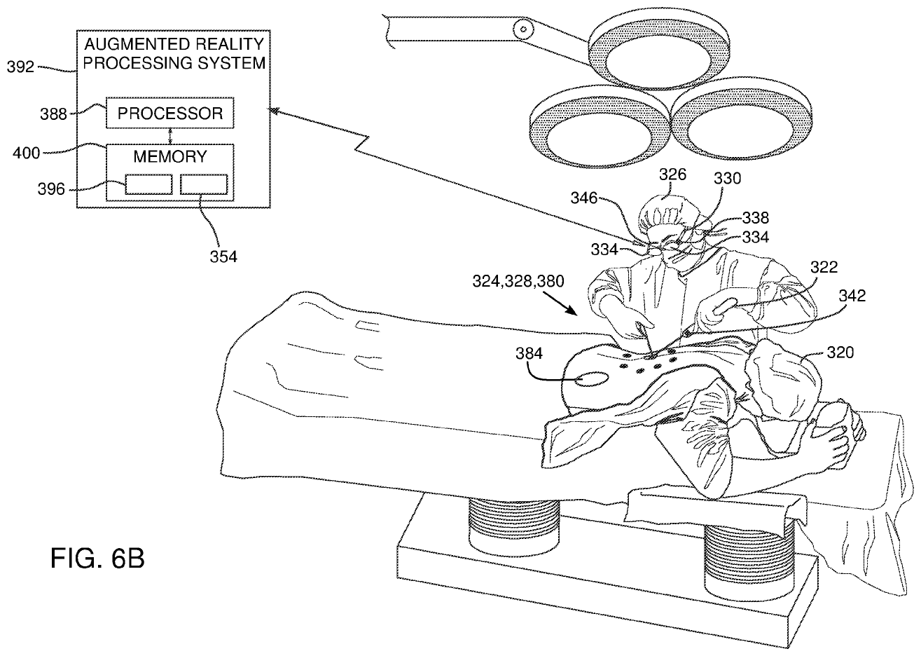

Reference is now made to FIGS. 6A and 6B, which are schematic illustrations of a registration phase of a medical procedure and of a medical procedure, respectively, that is performed upon a subject 320, also herein termed patient 320, using a set of fiducial landmarks, in accordance with an embodiment of the disclosure. In an initial stage of the procedure, a fiducial marker 324 is registered (e.g., via a CT or fluoroscope or other medical image scanner or imaging device), with patient 320. In the disclosed embodiment, fiducial marker 324 is comprised of a plurality of individual, fiducial landmarks 328A, 328B, 328C, generically termed fiducial landmarks 328.

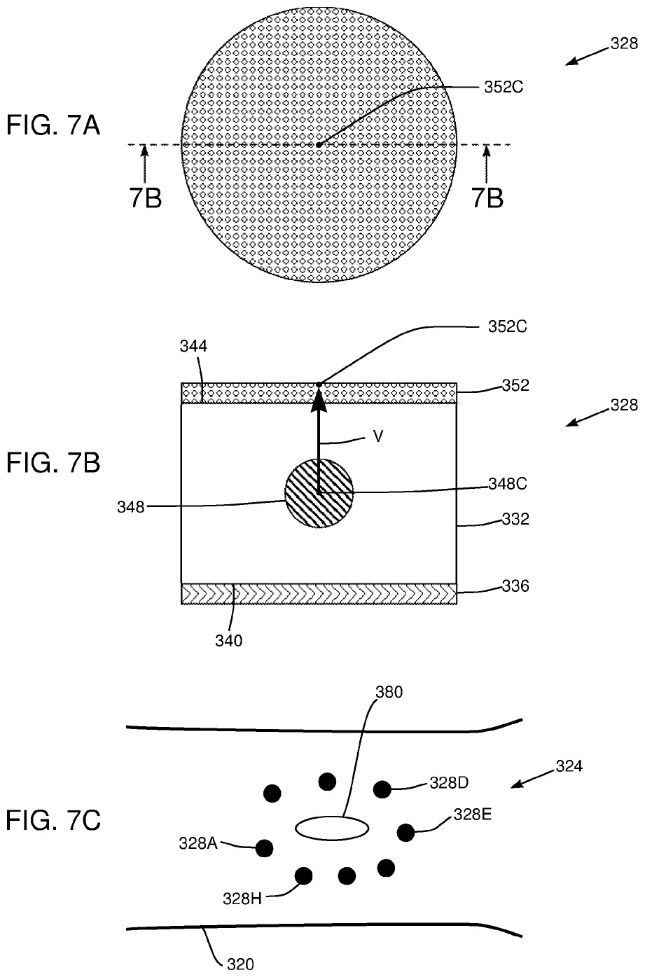

FIGS. 7A, 7B, and 7C respectively illustrate a top view and a cross-section view of fiducial landmarks 328, and a top view of a group of fiducial landmarks 328 forming marker 324, according to embodiments of the disclosure. The cross-section view is taken along a line 7B-7B of the top view. Marker 324 is shown as having eight fiducial landmarks 328 which are not coupled to each other. The number of fiducial landmarks may vary. For example, the number of fiducial landmarks may be between 8 and 12, 2, 3, 4, 5, 6, 7, 8, 9, 10, 11, 12, or more than 12. In some embodiments, fiducial landmarks 328 comprise a radiotransparent plane plate 332. The plate 332 may also be opaque to optical radiation. In the illustrated embodiment, plate 332 is formed as a circular disk. In one embodiment, the plate has an approximate diameter of 1 cm and an approximate thickness of 5 mm, and is produced from a plastic such as a polyimide. However, in other embodiments the size and the shape of plate 332 may be different e.g., a plate having an area that is more or less than π(0.5)² cm², and/or by having a thickness different from 5 mm, and/or by having a non-circular shape such as a rectangle. In some embodiments, the area of plate 332 is in the approximate range of 0.5 cm²-5 cm², although some embodiments may have areas outside this range.

In some embodiments, a biocompatible adhesive layer 336 is formed on a lower surface 340 of plate 332. Layer 336 may have similar adhesive properties to the adhesive layer of Band-Aid® adhesive bandages or surgical tape, thereby enabling plate 332 to be removably attached to the skin of patient 320. In some embodiments, layer 336 is formed from an acrylate such as a methacrylate or an epoxy diacrylate.

In some embodiments, a radiopaque element 348, hereinbelow also referred to as bead 348, having a centroid 348C, is incorporated within plate 332, and may be located at a center of symmetry of the plate. In some embodiments, bead 348 is approximately spherical, with a diameter of approximately 2 mm, but in other embodiments, the bead 348 has a different shape and/or a different diameter (e.g., a diameter between 0.5 mm and 5 mm, between 1 mm and 3 mm, between 1.5 mm and 2.5 mm, between 2 mm and 5 mm, overlapping ranges thereof, or any value within the recited ranges). In some embodiments, bead 348 is symmetrical, for example cylindrical or ellipsoidal, having a calculable centroid.

In some embodiments, an optical retroreflector 352, having a centroid 352C, coats an upper surface 344 of plate 332. Retroreflector 352 may be radiotransparent, and, in some embodiments, the retroreflector 352 comprises a retroreflective sheet that is cemented or otherwise adhered or fixed to upper surface 344 of plate 332.

As is illustrated in FIG. 7B, centroids 348C and 352C are spatially separated from each other, and a line segment joining the centroids 348C, 352C is orthogonal to surface 344. It will be understood that the spatial separation of the centroids can be characterized by a local centroid separation vector “V” that depends on the dimensions of plate 332, and on the positioning of bead 348. For example, if bead 348 is symmetrically located in plate 332, and the plate 332 has a thickness of 5 mm, then a local centroid separation vector V, that is 2.5 mm long, that is orthogonal to surface 344, and that initiates at centroid 348C, characterizes the centroid separation.

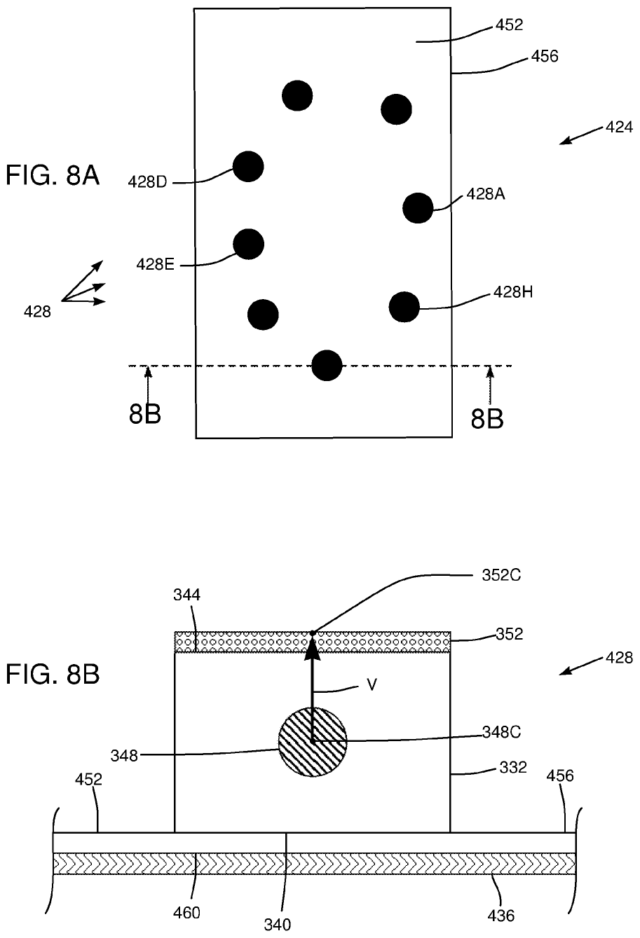

FIGS. 8A and 8B respectively illustrate a top view of a fiducial marker 424, and a cross-section view of a fiducial object 428 in the marker 424, according to an embodiment of the disclosure. The cross-section view is taken along a line 8B-8B of the top view. Fiducial marker 424 comprises a plurality of fiducial objects 428. Apart from the differences described below, the operation of fiducial marker 424 and fiducial objects 428 is generally similar to that of fiducial landmarks 324 acting as fiducial marker 324 and fiducial objects 328 (FIGS. 6A, 6B, 7A, 7B, and 7C), and elements indicated by the same reference numerals in fiducial markers 324 and 424 and fiducial objects 328 and 428 are generally similar in construction and in operation.

As for marker 324, marker 424 is comprised of a plurality of fiducial objects 428. However, in contrast to marker 324, the fiducial objects of marker 424 are coupled together, by being fixedly attached in a preset pattern to an upper surface 452 of a flexible radiotransparent sheet 456, which has a lower surface 460. Furthermore, and in contrast to fiducial objects 328, each fiducial object 428 does not have an adhesive layer 336 formed on lower surface 340 of plate 332. Rather, as shown in FIG. 8B, lower surface 340 of each fiducial object 428 is directly attached to upper surface 452 of sheet 456.

An adhesive layer 436 is formed on lower surface 460 of sheet 456, the adhesive of the layer having similar properties to the adhesive of layer 336, described above.

It will be appreciated that in contrast to fiducial marker 324, where fiducial objects 328 are not coupled together and are independently located in a random pattern with respect to each other, in fiducial marker 424, fiducial objects 428 are coupled together in a preset pattern by being fixedly attached to sheet 456. Thus, in marker 324, the fiducial objects 328 of the marker 324 may be individually attached to the skin of patient 320, and in marker 424, the marker 424 acts as a “sticker” which, in one operation, can be attached to the skin of the patient.

The following description assumes, for simplicity and except where otherwise indicated, that fiducial marker 324 is used in the procedure illustrated in FIGS. 6A and 6B. Those having ordinary skill in the art will be able to adapt the description, mutatis mutandis, if fiducial marker 424 is used.

Returning to FIG. 6A, in the illustrated initial stage of the procedure wherein fiducial marker 324 is registered, a plurality of fiducial landmarks 328 of the marker 324 are attached to the skin of patient 320, in proximity to a site 380 of the procedure (e.g., surgical or other medical procedure). In the description herein, the procedure is performed by a surgeon 326 and is assumed, by way of example, to be on a spine of patient 320. Those having ordinary skill in the art will be able to adapt the description, mutatis mutandis, for other procedures using registration of a marker, such as for iliac surgery, sacroiliac joint procedures, joint replacement procedures, bone cut procedures, discectomy procedures, heart surgery procedures, arthroscopic procedures, cranial procedures, cardiovascular procedures, tissue repair procedures, and/or the like.

Fiducial landmarks 328 may be attached, by their adhesive layer 336, to the patient's skin so as to surround site 380.

Once fiducial landmarks 328 have been attached to patient 320 a medical imaging device (e.g., a computerized tomography (CT) device or fluoroscope 386), is used to scan patient 320. The scan may be used to acquire a medical image (e.g., CT or fluoroscopic image) 396 of fiducial landmarks 328 and patient 320, and a processor 388 of an augmented reality processing system 392 used by surgeon 326 may store the acquired image in a memory 400 of the system. As is explained further below, processor 388 may be configured to analyze the image to register patient 320 with fiducial marker 324.

Referring now to FIG. 6B, in a subsequent stage of the procedure, where fiducial marker 324 is tracked, using optical radiation, and the tracking, using the registration of the initial stage, is used, inter alia, to compensate for any relative movement between surgeon 326 performing the procedure, and patient 320.

In the subsequent stage, surgeon 326 may use a tool 322 to perform an action with respect to the patient's back or other anatomical portion of the patient 320, the tool 322 being inserted via an incision on the patient's back or other anatomical location at site 380.

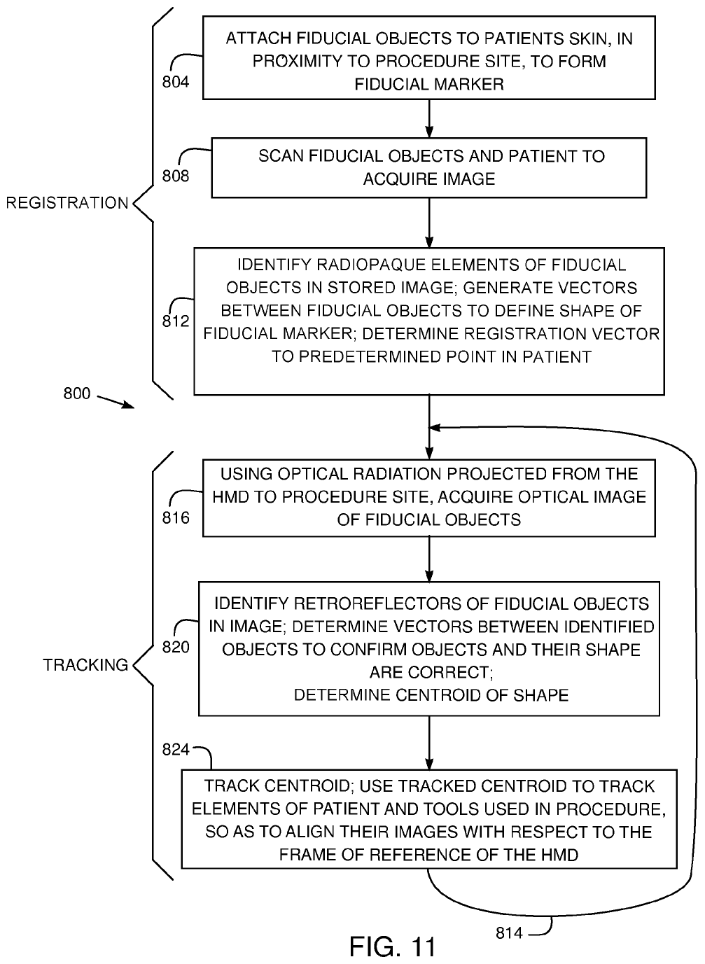

FIG. 11 is a flowchart 800 of steps that may be performed in implementing a registration/tracking algorithm 354, according to an embodiment of the disclosure. The algorithm is assumed to be performed during the medical procedure illustrated in FIGS. 6A and 6B, wherein surgeon 326 performs the procedure. In the following description, except as stated otherwise below, fiducial objects 328, forming fiducial marker 324, are assumed, by way of example, to be used for the registration and the tracking; however, fiducial marker 424 may also be used. The registration/tracking algorithm 354 may be performed by performing program instructions or tasks executed by any one or more of the processors described herein (e.g., processor 26, 388, 545, 724).

In an initial step 804, a plurality of fiducial objects 328, forming fiducial marker 324, are attached to patient 320. The attachment uses the respective adhesive layers of the fiducial objects 328 to adhere the objects 328 to the skin of patient 320. In some embodiments, the objects 328 are placed in a random pattern on the patient's skin. The fiducial objects are placed in proximity to site 380 of the procedure. In some implementations, the fiducial objects 328 are positioned to surround site 380. In some implementations, the fiducial objects 328 are positioned in localized region 384 (shown in FIG. 6B).

In a scan step 808, patient 320 and the attached fiducial objects 328 are scanned, (e.g., by a cone beam computerized tomography (CBCT) fluoroscope or other fluoroscope or medical imaging device). The image 396 generated by the scan may be stored (e.g., by processor 388) in memory (e.g., memory 400) of the augmented reality processing system.

In an analysis step 812, processor 388 analyzes the stored image to identify images and locations of radiopaque elements (e.g., radiopaque beads 348) in the fiducial objects 328. Once identified, the processor generates (e.g., determines, accesses, calculates) a set of local vectors between determined (e.g., calculated) centroids (e.g., centroids 348C, 352C) of the identified radiopaque elements (e.g., beads 348. In some embodiments, the set of vectors is descriptive of the shape of the random pattern of the attached fiducial objects 328.

In addition, processor 388 determines (e.g., formulates, generates, calculates) a local registration vector between a point in the set of radiopaque elements (e.g., beads 348) and a point, such as the origin, in the coordinate system of the scan volume (e.g., fluoroscopic scan volume) of the patient. In some embodiments, processor 388 determines the location of a centroid of the set of radiopaque elements (e.g., beads 348), and formulates a local registration vector between the centroid of the set of radiopaque elements and a predetermined patient point, such as one of the vertebrae of the patient. Step 812 concludes the registration stage of the flowchart 800.

The following steps of the flowchart 800 correspond to the tracking stage of the procedure, and these steps are reiterated during the procedure, as shown by an arrow 814.

In an optical imaging step 816, which is the initial step of the tracking stage, surgeon 326 is assumed to wear HMD 330. Light source 346 (e.g., projector) of the HMD 330 projects optical radiation (e.g., infrared radiation) as described hereinabove, to site 380 and its surroundings, and camera 338 of the HMD 330 acquires an optical image of the irradiated region. In some embodiments, processor 388 stores the optical image acquired by camera 338 in memory 400.

In an analysis step 820, processor 388 analyzes the stored optical image to identify retroreflectors 352 of fiducial objects 328. The processor 388 determines (e.g., calculates) a set of local vectors between centroids (e.g., centroids 348C, 352C) of the identified retroreflectors 352, and compares this set of local vectors with the set of vectors generated in analysis step 812 in the registration phase. The comparison may be performed by, for example finding differences between respective vectors of the two sets, and assuming that the two sets are for a common group of fiducial objects if the total of the differences is below a preset value. Other methods of performing the comparison may also be used.

If the analysis at step 820 indicates that the two sets of vectors are for a common group, e.g., represent a common shape, then the processor 388 determines (e.g., calculates) a location of the centroid of the shape of the identified retroreflectors 352. The processor 388 applies the local centroid separation vector “V”, described above with reference to FIG. 8B, to the retroreflector centroid to calculate a location for the radiopaque element (e.g. bead) centroid. The separation vector V may be applied to centroid of the retroreflectors 352 because fiducial objects 328 have a common construction, and have substantially the same orientation.

In some instances, during the tracking, at least one fiducial object 328 may have moved relative to the other fiducial objects 328 forming marker 324. Alternatively or additionally, at least one of retroreflectors 352 may be obscured or may be imaged poorly. Processor 388 may be configured to check for these cases, and to identify the objects and the retroreflectors 352 of the cases. The check may be performed by finding that there is a subset of “outlying” vectors, e.g., vectors that differ by more than a preset value from the local vectors calculated in registration analysis step 812, in the calculated set of local vectors. Such outlying vectors may be generated by the moved objects or obscured retroreflectors, enabling the processor 388 to identify these objects.

If processor 388 has determined there are moved objects 328, or objects 328 having poorly imaged retroreflectors 352, then the processor 388 may use the locations of objects 328 that have not moved and that have well-imaged retroreflectors, making allowances for the moved or poorly imaged objects, to calculate an effective centroid for the retroreflectors 352.

In some cases, the comparison of the two sets of vectors may exceed the preset value. Such a case typically occurs if a large number of objects 328 have moved. In this case, processor 388 may provide a notification to surgeon 326 that tracking is paused.

In a tracking step 824, the processor 388 uses the location of the radiopaque element centroid (e.g., bead centroid) found in step 820, together with the local registration vector formulated in analysis step 812, to track elements of patient 320, and tools such as tool 322. In accordance with several embodiments, because the tracking uses images acquired by camera 338, which is fixed to HMD 330, the processor 388 is able to determine locations and orientations of the patient elements and of the tools in the frame of reference of the HMD 330, and thus to present correctly aligned images on the displays of the HMD 330.

The disclosed systems, methods, software products, hardware elements and/or functionality described with respect to at least one of HMD 24, HMD 537, HMD 700 or HMD 330 may apply, mutatis mutandis, to any one of the other HMDs. Any display described with respect to at least one of display 70, display 56a, display 530, display 720 or display 334, may apply, mutatis mutandis, to any one of the other displays. The disclosed systems, methods, software products, hardware elements and/or functionality described with respect to at least one of system 20 or system 392 may apply, mutatis mutandis, to the other system. The disclosed systems, methods, software products, hardware elements and/or functionality described with respect to at least one of processors 26, 388, 545, 724 may apply, mutatis mutandis, to any one of the other processors.

The processors 26, 388, 545, 724 may include one or more central processing units (CPUs) or processors, which may each include a conventional or proprietary microprocessor. The processors 26, 388, 545, 724 may be communicatively coupled to one or more memory units, such as random-access memory (RAM) for temporary storage of information, one or more read only memory (ROM) for permanent storage of information, and one or more mass storage devices, such as a hard drive, diskette, solid state drive, or optical media storage device. The processors 26, 388, 545, 724 (or memory units communicatively coupled thereto) may include modules comprising program instructions or algorithm steps configured for execution by the processors 26, 388, 545, 724 to perform any of all of the processes or algorithms discussed herein. The processors 26, 388, 545, 724 may be communicatively coupled to external devices (e.g., display devices, data storage devices, databases, servers, etc. over a network via a network communications interface.

Each of the processes, methods, and algorithms described in the preceding sections may be embodied in, and fully or partially automated by, code modules executed by one or more computer systems or computer processors comprising computer hardware. The methods may be executed on the computing devices in response to execution of software instructions or other executable code read from a tangible computer readable medium. A tangible computer readable medium is a data storage device that can store data that is readable by a computer system. The code modules may be stored on any type of non-transitory computer-readable medium or computer storage device, such as hard drives, solid state memory, optical disc, and/or the like. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process steps may be stored, persistently or otherwise, in any type of non-transitory computer storage such as, for example, volatile or non-volatile storage.

In general, the word “module,” as used herein, refers to logic embodied in hardware or firmware, or to a collection of software instructions, possibly having entry and exit points, written in a programming language, such as, for example, Java, Lua, C, C #, or C++. A software module or product may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules described herein may be implemented as software modules but may additionally or alternatively represented in hardware or firmware. Generally, any modules or programs or flowcharts described herein may refer to logical modules that may be combined with other modules or divided into sub-modules despite their physical organization or storage.

The various features and processes described above may be used independently of one another or may be combined in various ways. All possible combinations and sub-combinations are intended to fall within the scope of this disclosure. In addition, certain method or process blocks or steps may be omitted in some implementations. The methods and processes described herein are also not limited to any particular sequence, and the blocks, steps, or states relating thereto can be performed in other sequences that are appropriate. For example, described blocks, steps, or states may be performed in an order other than that specifically disclosed, or multiple blocks or states may be combined in a single block or state. The example blocks, steps, or states may be performed in serial, in parallel, or in some other manner. Blocks, steps, or states may be added to or removed from the disclosed example embodiments. The example systems and components described herein may be configured differently than described. For example, elements may be added to, removed from, or rearranged compared to the disclosed example embodiments.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

Although the drawings relate specifically to surgery on the spine, the principles of the present disclosure may similarly be applied in other procedures, such as but not limited to arthroscopic procedures (including joint replacement, such as hip replacement, knee replacement, shoulder joint replacement or ankle joint replacement; reconstructive surgery (e.g., hip surgery, knee surgery, ankle surgery, foot surgery); joint fusion surgery; laminectomy; osteotomy; neurologic surgery (e.g., brain surgery, spinal cord surgery, peripheral nerve procedures); ocular surgery; urologic surgery; cardiovascular surgery (e.g., heart surgery, vascular intervention); oncology procedures; biopsies; tendon or ligament repair; and/or organ transplants.

In some embodiments, the system comprises various features that are present as single features (as opposed to multiple features). For example, in one embodiment, the system includes a single HMD, a single camera, a single processor, a single display, a single fiducial marker, a single imaging device, etc. Multiple features or components are provided in alternate embodiments.

In some embodiments, the system comprises one or more of the following: means for imaging (e.g., a camera or fluoroscope or MRI machine or CT machine), means for calibration or registration (e.g., adapters, markers, objects), means for fastening (e.g., anchors, adhesives, clamps, pins), etc.

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure.

It should be noted that the use of particular terminology when describing certain features or aspects of the systems and methods should not be taken to imply that the terminology is being re-defined herein to be restricted to including any specific characteristics of the features or aspects of the systems and methods with which that terminology is associated. While the embodiments provide various features, examples, screen displays, user interface features, and analyses, it is recognized that other embodiments may be used.

The drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure. The section headings used herein are merely provided to enhance readability and are not intended to limit the scope of the embodiments disclosed in a particular section to the features or elements disclosed in that section.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

The terms “comprising.” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise.

The terms “top,” “bottom,” “first,” “second,” “upper,” “lower,” “height,” “width,” “length,” “end,” “side,” “horizontal,” “vertical.” and similar terms may be used herein; it should be understood that these terms have reference only to the structures shown in the figures and are utilized only to facilitate describing embodiments of the disclosure. Various embodiments of the disclosure have been presented in a range format. It should be understood that the description in range format is merely for convenience and brevity and should not be construed as an inflexible limitation on the scope of the invention. The ranges disclosed herein encompass any and all overlap, sub-ranges, and combinations thereof, as well as individual numerical values within that range. Language such as “up to.” “at least,” “greater than.” “less than,” “between,” and the like includes the number recited. Numbers preceded by a term such as “about” or “approximately” include the recited numbers. For example, “approximately 2 mm” includes “2 mm.” The terms “approximately”, “about”, and “substantially” as used herein represent an amount close to the stated amount that still performs a desired function or achieves a desired result.

As used herein “generate” or “generating” or “determine” or “determining” may include specific algorithms for creating information based on or using other input information. Generating or determining may include retrieving the input information such as from memory or as provided input parameters to the hardware performing the generating or determining. Once obtained, the generating or determining may include combining the input information. The combination may be performed through specific circuitry configured to provide an output indicating the result of the generating or determining. The combination may be dynamically performed such as through dynamic selection of execution paths based on, for example, the input information, device operational characteristics (for example, hardware resources available, power level, power source, memory levels, network connectivity, bandwidth, and the like). Generating or determining may also include storing the generated or determined information in a memory location. The memory location may be identified as part of the request message that initiates the generating or determining. In some implementations, the generating or determining may return location information identifying where the generated or determined information can be accessed. The location information may include a memory location, network locate, file system location, or the like. Generating or determining may include calculating by one or more processors. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A method, comprising:
obtaining a transformation for registering a representation of internal anatomy of a subject with a coordinate system with respect to which a fiducial, which is coupled to the subject, is tracked;
by applying the transformation:
computing initial coordinates, relative to the representation, of one or more landmarks on skin of the subject,
facilitating a procedure on the subject by displaying the representation in registration with the coordinate system, and
computing subsequent coordinates of the landmarks relative to the representation;
computing one or more distances between the subsequent coordinates and the initial coordinates; and in response to the distances, generating an output.

2. The method according to claim 1, wherein the fiducial is tracked by identifying the fiducial in tracking images acquired by a camera.

3. The method according to claim 1,
wherein the procedure is performed by a physician viewing the subject through a near-eye display of a head-mounted display device, and
wherein displaying the representation comprises displaying the representation on the near-eye display so as to augment the view of the physician.

4. The method according to claim 1, wherein a distance from the fiducial to each of the landmarks is at least ten cm.

5. The method according to claim 1,
wherein the fiducial is a first fiducial,
wherein the procedure is performed using a tool,
wherein a second fiducial, which is tracked with respect to the coordinate system, is coupled to the tool, and
wherein displaying the representation comprises displaying the representation with an overlaid virtual representation of the tool at a portion of the representation corresponding to a location of the tool.

6. The method according to any one of claims 1 to 5, wherein displaying the representation comprises displaying the representation with respective virtual representations of the landmarks at the initial coordinates, respectively, and wherein generating the output comprises modifying at least some of the virtual representations of the landmarks.

7. The method according to any one of claims 1 to 5, wherein generating the output comprises generating the output in response to one or more of the distances exceeding a predefined threshold.

8. The method according to claim 7, wherein the output indicates that those of the landmarks corresponding to those of the distances that exceed the threshold should not be used for subsequent validation of the transformation.

9. The method according to claim 7, wherein generating the output comprises:
selecting one or more of the landmarks corresponding to those of the distances that do not exceed the threshold; and
generating the output so as to indicate that the selected ones of the landmarks should be used for subsequent validation of the transformation.

10. The method according to claim 9, wherein each of the selected ones of the landmarks is farther from a closest one of unselected ones of the landmarks than is any other one of the unselected ones of the landmarks.

11. The method according to claim 7, wherein generating the output comprises generating the output so as to indicate that one or more of the landmarks corresponding to those of the distances that exceed the threshold may have locally shifted.

12. The method according to claim 11,
wherein the threshold is a first threshold.
wherein the method further comprises computing respective initial pairwise distances between pairs of the landmarks, and
wherein generating the output so as to indicate that one or more landmarks may have locally shifted comprises:
computing respective subsequent pairwise distances between the pairs; and
generating the output so as to indicate that the local shift may have occurred in response to a magnitude of a difference between one of the subsequent pairwise distances and a corresponding one of the initial pairwise distances exceeding a second predefined threshold.

13. The method according to any one of claims 1 to 5, wherein the output indicates that the transformation may have become invalid.

14. The method according to any one of claims 1 to 5,
wherein the fiducial is a first fiducial,
wherein a second fiducial, which is coupled to a tool, is tracked with respect to the coordinate system while the tool contacts different respective ones of the landmarks, and
wherein computing the subsequent coordinates comprises:
in response to the tracking of the second fiducial, computing respective base coordinates of the landmarks; and
computing the subsequent coordinates by transforming the base coordinates per the transformation.

15. The method according to any one of claims 1 to 5, wherein the computing of the one or more distances between the subsequent coordinates and the initial coordinates comprises for each subsequent coordinate, computing a distance between the subsequent coordinate and each of the initial coordinates.

16. The method according to claim 15, wherein a landmark is determined valid if a distance between its subsequent coordinates and initial coordinates does not exceed a predefined threshold.

17. The method according to any one of claims 1 to 5, wherein the computing of the initial exceeding a predefined threshold coordinates of the landmarks and the computing of the subsequent coordinates of the landmarks are performed are performed according to instructions provided by a user.

18. The method according to any one of claims 1 to 5, wherein the landmarks are uniquely identified by a characteristic and wherein the method further comprises storing the initial coordinates of each landmark in association with its uniquely identifying characteristic.

19. The method according to any one of claims 1 to 5, wherein the method further comprises, for each landmark subsequent coordinates, a user input indicating the corresponding landmark initial coordinates.

20. The method according to any one of claims 1 to 5, wherein each landmark comprises one or more uniquely identifying retroreflective elements.

21. A method for tracking a transformation during a medical procedure, the transformation registering a representation of internal anatomy of a subject with a coordinate system with respect to a fiducial, the fiducial being coupled to the subject, the method comprising:
determining initial coordinates of one or more landmarks relative to the representation, the one or more landmarks being disposed on skin of the subject;
displaying the representation in registration with the coordinate system;
determining subsequent coordinates of the one or more landmarks relative to the representation;
determining one or more distances between the subsequent coordinates and the initial coordinates; and
determining whether the registration is valid based at least in part on the one or more distances.

22. The method according to claim 21, wherein the fiducial is tracked by identifying the fiducial in tracking images acquired by a camera.

23. The method according to claim 21, wherein the medical procedure is performed by a physician viewing the subject through a near-eye display, and wherein displaying the representation comprises displaying the representation on the near-eye display so as to augment the view of the physician.

24. The method according to claim 21, wherein a distance from the fiducial to each of the one or more landmarks is at least ten cm.

25. The method according to claim 21, wherein the fiducial is a first fiducial, wherein the procedure is performed using a tool, wherein a second fiducial, which is tracked with respect to the coordinate system, is coupled to the tool, and wherein displaying the representation comprises displaying the representation with an overlaid virtual representation of the tool at a portion of the representation corresponding to a location of the tool.

26. The method according to claim 21, wherein displaying the representation comprises displaying the representation with respective virtual representations of the one or more landmarks at the initial coordinates, respectively, and wherein determining whether the registration is valid comprises modifying at least some of the virtual representations of the landmarks.

27. The method according to any one of claims 21 to 26, wherein determining whether the registration is valid comprises determining whether the one or more distances exceed a predefined threshold.

28. The method according to claim 27, wherein the one or more landmarks corresponding to the one or more distances that exceed the predefined threshold are not used for subsequent validation of the transformation.

29. The method according to claim 27, wherein the one or more landmarks corresponding to the one or more distances that do not exceed the predefined threshold are used for subsequent validation of the transformation.

30. The method according to claim 27, further comprising identifying the one or more landmarks corresponding to the one or more distances that exceed the predefined threshold may have locally shifted.

31. The method according to claim 30, wherein the predetermined threshold is a first threshold, and wherein identifying the one or more landmarks that may have shifted comprises:
determining respective initial pairwise distances between pairs of the one or more landmarks;
determining respective subsequent pairwise distances between the pairs of the one or more landmarks;
determining a magnitude of a difference between one of the subsequent pairwise distances and a corresponding one of the initial pairwise distances; and
comparing the magnitude to a second predefined threshold.

32. The method according to any one of claims 21 to 26, wherein the fiducial is a first fiducial, wherein a second fiducial, which is coupled to a tool, is tracked with respect to the coordinate system while the tool contacts different respective ones of the one or more landmarks, and wherein determining the subsequent coordinates comprises:
in response to the tracking of the second fiducial, determining respective base coordinates of the one or more landmarks; and
determining the subsequent coordinates based at least in part on the one or more distances.

33. The method according to any one claims 21 to 26, wherein determining the one or more distances between the subsequent coordinates and the initial coordinates comprises for each subsequent coordinate, determining a distance between the subsequent coordinate and each of the initial coordinates.

34. The method according to claim 33, wherein the one or more landmarks are determined valid if a distance between its subsequent coordinates and initial coordinates does not exceed a predefined threshold.

35. The method according to any one of claims 21 to 26, wherein the one or more landmarks are uniquely identified by a characteristic, and wherein the method further comprises storing the initial coordinates of the one or more landmarks in association with its uniquely identifying characteristic.

36. The method according to any one of claims 21 to 26, wherein the one or more landmarks comprise one or more uniquely identifying retroreflective elements.

37. A system for tracking a transformation during a medical procedure, the transformation registering a representation of internal anatomy of a subject with a coordinate system with respect to one or more fiducial objects coupled to the subject, the system comprising:
a head-mounted display device comprising a near-eye display and a tracking system;
a plurality of landmarks forming the one or more fiducial objects, the plurality of landmarks configured to be disposed on skin of the subject in proximity to a site of the medical procedure; and
one or more processors, that upon execution of program instructions stored on a non-transitory computer-readable medium:
determine initial coordinates of the plurality of landmarks relative to the representation based on one or more images received by the tracking system of the plurality of landmarks and the site of the medical procedure;
display the representation in registration with the coordinate system on the near-dye display of the head-mounted device;
determine subsequent coordinates of the plurality of landmarks relative to the representation;
determine one or more distances between the subsequent coordinates and the initial coordinates; and
determine whether the registration is valid based at least in part on the one or more distances.

38. The system of claim 37, wherein the head-mounted display device comprises a pair of glasses.

39. The system of claim 37, wherein the tracking system comprises an infrared camera.

40. The system of claim 39, wherein the tracking system further comprises a projector configured to project infrared light toward the site of the medical procedure.

41. The system of claim 37, wherein the plurality of landmarks comprise registration markers.

42. The system of any one of claims 37 to 41, wherein the plurality of landmarks comprise adhesive stickers.

43. The system of any one of claims 37 to 41, wherein the plurality of landmarks comprise one or more uniquely identifying retroreflective elements.

44. The system of any one of claims 37 to 41, wherein the plurality of landmarks are disposed in a random pattern on the skin of the subject.

45. The system of any one of claims 37 to 41, wherein the plurality of landmarks comprise one or more radiopaque elements.

46. A fiducial object, comprising:
a radiotransparent plate having a first surface coated with an optical retroreflector, and a second surface opposite the first surface;
a radiopaque element incorporated within the radiotransparent plate; and
an adhesive layer, formed on the second surface, configured to removably adhere to skin of a human subject.

47. The fiducial object according to claim 46, wherein the radiopaque element is a preset distance from the first surface.

48. The fiducial object according to claim 46, wherein the optical retroreflector is radiotransparent.

49. The fiducial object according to claim 46, wherein the radiopaque element comprises a radiopaque bead having a symmetrical shape.

50. A fiducial marker, comprising:
a flexible sheet, having a first sheet surface and a second sheet surface opposite the first sheet surface;
a plurality of fiducial objects, each fiducial object comprising:
a radiotransparent plate having a first plate surface coated with an optical retroreflector, and a second plate surface, opposite the first plate surface, affixed to the first sheet surface;
a radiopaque element incorporated within the radiotransparent plate; and
an adhesive layer, formed on the second sheet surface, configured to removably adhere to skin of a human subject.

51. The fiducial marker according to claim 50, wherein the plurality of fiducial objects are affixed to the first sheet surface in a preset pattern.

52. A fiducial marker, comprising:
a plurality of fiducial objects, each fiducial object comprising:
a radiotransparent plate having a first surface coated with an optical retroreflector, and a second surface opposite the first surface;
a radiopaque element incorporated within the radiotransparent plate; and
an adhesive layer, formed on the second surface, configured to removably adhere to skin of a human subject.

53. A method for registering a plurality of fiducial objects, individually attached to the skin of a patient, with the patient, each of the fiducial objects comprising a radiopaque element, the method comprising:
accessing a fluoroscopic image of the fiducial objects;
identifying, in each of the fiducial objects, respective locations of the radiopaque element therein; and
in response to the identified respective locations, formulating a vector between a selected point of the fiducial objects and the patient, so as to register the fiducial objects with the patient.

54. The method according to claim 53, wherein the vector is between a centroid of the fiducial objects and a vertebra of the patient.

55. The method according to claim 53, wherein the vector is between a selected one of the fiducial objects and a point in a fluoroscopic scan providing the fluoroscopic image.

56. A method for tracking a plurality of fiducial objects individually attached to the skin of a patient, each of the fiducial objects comprising a radiopaque element therein and a retroreflector thereon, the method comprising:
accessing a fluoroscopic image of the fiducial objects;
identifying, in each of the fiducial objects, respective locations of the radiopaque elements therein;
in response to the identified respective locations, defining a first shape of the attached fiducial objects;
acquiring an optical image of the fiducial objects in response to optical radiation transmitted from a head mounted display and identifying the retroreflectors in the image;
formulating a second shape of the attached fiducial objects in response to the identified retroreflectors; and
when the second shape corresponds to the first shape, using the identified retroreflectors to track the plurality of fiducial objects in a frame of reference of the head mounted display.

57. The method according to claim 56, wherein defining the first shape comprises generating a set of local vectors between the identified locations of the radiopaque elements.

58. The method according to claim 56, wherein formulating the second shape comprises generating a set of local vectors between locations of the identified retroreflectors.

59. A method for tracking a patient in a frame of reference of a head-mounted display, the patient having a plurality of fiducial objects individually attached thereto, each of the fiducial objects comprising a radiopaque element therein and a retroreflector thereon, the method comprising:
accessing a fluoroscopic image of the fiducial objects;
identifying, in each of the fiducial objects, respective locations of the radiopaque element therein;
in response to the identified respective locations, defining a first shape of the attached fiducial objects and formulating a vector between a selected point of the fiducial objects and the patient, so as to register the fiducial objects with the patient;
acquiring an optical image of the fiducial objects in response to optical radiation transmitted from the head mounted display, and identifying the retroreflectors in the optical image;
formulating a second shape of the attached fiducial objects in response to the identified retroreflectors; and
when the second shape corresponds to the first shape, using the identified retroreflectors to track the patient in the frame of reference of the head mounted display.
